(19) United States
(12) Patent Application Publication
(10) Pub. No.: US 2015/0274891 A1
(43) Pub. Date: Oct. 1, 2015

(54) MEMBRANES WITH IMPROVED FLUX AND METHOD FOR THEIR PREPARATION

(71) Applicant: BASF SE, Ludwigshafen (DE)

(72) Inventors: Rupert Konradi, Ladenburg (DE); Kristine Hartnagel, Lorsch (DE)

(73) Assignee: BASF SE, Ludwigshafen (DE)

(21) Appl. No.: 14/435,268

(22) PCT Filed: Dec. 16, 2013
(86) PCT No.: PCT/EP2013/076751

§ 371 Date: Apr. 13, 2015

Related U.S. Application Data
(60) Provisional application No. 61/737,870, filed on Dec. 17, 2012.
(60) Provisional application No. 61/737,942, filed on Dec. 17, 2012.
(60) Provisional application No. 61/737,882, filed on Dec. 17, 2012.

(30) Foreign Application Priority Data
Dec. 17, 2012 (EP) 12197526.2
Dec. 17, 2012 (EP) 12197556.9
Dec. 17, 2012 (EP) 12197599.9
Dec. 17, 2012 (EP) 12197613.8

(51) Int. Cl. C08G 73/06; B01D 71/62; B01D 71/12; B01D 67/00; B01D 61/00; B01D 61/14; C02F 1/44; A61M 1/16; C09D 179/04; B01D 61/08

(52) U.S. Cl. CPC C08G 73/06; C09D 179/04; B01D 71/62; B01D 71/12; B01D 61/08; B01D 61/002; B01D 61/145; B01D 61/147; C02F 1/44; A61M 1/1621; B01D 67/0002; B01D 67/0093; B01D 67/009; C02F 2103/08

(57) ABSTRACT

Polymer, comprising i) at least one oxazoline according to formula wherein R¹, R², R³ and R⁴ independently denote a hydrogen atom, a halogen atom, an alkyl group, an aralkyl group, a phenyl group, or a substituted phenyl group, and R⁵ denotes a noncyclic organic group having an unsaturated bond reactive in radical polymerization, ii) optionally at least one antiadhesive monomer, iii) optionally at least one biocidal monomer, iv) optionally at least one further monomer, wherein said polymer comprises at least one monomer selected from monomers ii) and iii).

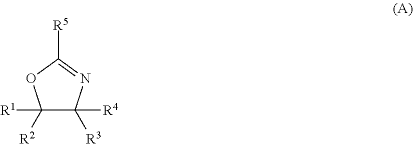

The present invention relates to polymers comprising:
i) at least one oxazoline according to formula

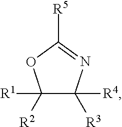

wherein R¹, R², R³ and R⁴ independently denote a hydrogen atom, a halogen atom, an alkyl group, an aralkyl group, a phenyl group, or a substituted phenyl group, and R⁵ denotes a noncyclic organic group having an unsaturated bond reactive in radical polymerization,
ii) optionally at least one antiadhesive monomer,
iii) optionally at least one biocidal monomer,
iv) optionally at least one further monomer,
wherein said polymer comprises at least one monomer selected from monomers ii) and iii).

The invention further relates to novel membranes, processes for making such membranes, the use of such membranes and to a method of increasing the flux through a membrane.

Different types of membranes play an increasingly important role in many fields of technology. In particular, methods for treating water rely more and more on membrane technology.

An important issue with the application of membranes is fouling. The problem of biofouling is pronounced in semipermeable membranes used for separation purposes like reverse osmosis, forward osmosis, nanofiltration, ultrafiltration and micro filtration. Membranes may be classified according to their separation mechanism and/or pore sizes. For example, in water filtration applications ultrafiltration and microfiltration membranes (approximate pore diameter: 5-1000 nm) are used for wastewater treatment retaining organic and bioorganic material. In reverse osmosis and forward osmosis membranes, where monovalent ions and all components with larger diameter are rejected, the separation mechanism is based mainly on solution-diffusion mechanism.

In all applications where the ambient medium is an aqueous phase, potential blockage may occur by adhesion of microorganisms and biofilm formation. As a consequence, a membrane is desired, which reduces biofilm formation and thus requires fewer cleaning cycles. This can for example be achieved through membranes with anti-adhesive or antifouling properties.

Thus, fouling is currently one of the major remaining problems for filtration membranes. Fouling causes deterioration of the membrane performance and shortens membrane lifetime, limiting further application of membrane technology. It is thus desirable to improve antifouling and antibacterial properties to membranes without impairing their separation characteristics in order to enhance their resistance.

Several approaches have been tried to solve the problem of fouling and biofouling and to prevent the formation and deposition of organic materials from organisms.

Recent research has focused on three strategies to prevent biofouling of membranes: 1) blending of hydrophilic or amphiphilic copolymers for the manufacture of membranes; 2) surface modification of membranes and 3) bulk modification of membrane materials.

The following documents describe approaches undertaken in recent years:
H. Yamamura, K. Kimmura, Y. Watanabe, Mechanism involved in the evolution of physically irreversible fouling in microfiltration and ultrafiltration membranes used for drinking water treatment, Environ. Sci. Technol. 41 (2007) 6789-6794.
V. Kochkodan, S. Tsarenko, N. Potapchenko, V. Kosinova, V. Goncharuk, Adhesion of microorganisms to polymer membranes: a photobactericidal effect of surface treatment with Ti02, Desalination 220 (2008) 380-385.
J. Mansouri, S. Harrisson, Vicki Chen, Strategies for controlling biofouling in membrane filtration systems: challenges and opportunities. J. Mater. Chem., 20 (2010).
U.S. Pat. No. 4,277,344 discloses antifouling approaches on RO layers, formed by interfacial reaction.
Desalination 275 (2011) 252-259, describes the grafting of PEG on a polyamide layer.
U.S. Pat. No. 6,280,853 and US 2010/043,733 disclose coatings of composite membranes with various polymers including polyalkylene oxide compounds or polyacrylamide compounds.
A. V. R. Reddy, D. J. Mohan, A. Bhattacharya, V. J. Shah, P. K. Ghosh, Surface modification of ultrafiltration membranes by preadsorption of a negatively charged polymer: I. Permeation of water soluble polymers and inorganic salt solutions and fouling resistance properties, J. Membr. Sci. 214 (2003) 211-221.
K. C. Khulbe, C. Feng, T. Matsuura, The art of surface modification of synthetic polymeric membranes, J. Appl. Polym. Sci. 115 (2010) 855-895.
B. Van der Bruggen, Chemical modification of polyethersulfone nanofiltration membranes: A review, J. Appl. Polym. Sci. 114 (2009) 630-642.
US 2007/0251883 discloses RO membranes with a branched polyalkyleneoxide modified antifouling surface.
EP 722 116, U.S. Pat. No. 5,254,664, JP 2001 49214 and U.S. Pat. No. 5,705,573 disclose polymeric mixtures comprising oxazolines.

It was one object of the invention, to provide novel polymers capable of increasing the flux through a membrane. In the context of this application, “improving the flux” shall also be understood to mean “reducing the decrease of flux through a membrane over time”. This objective has been achieved by a polymer, comprising
i) at least one oxazoline according to formula (O)

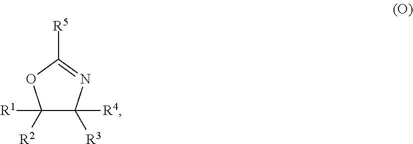

wherein R¹, R², R³ and R⁴ independently denote a hydrogen atom, a halogen atom, an alkyl group, an aralkyl group, a phenyl group, or a substituted phenyl group, and R⁵ denotes a noncyclic organic group having an unsaturated bond reactive in radical polymerization,
ii) optionally at least one antiadhesive monomer,
iii) optionally at least one biocidal monomer,
iv) optionally at least one further monomer,
wherein said polymer comprises at least one monomer selected from monomers ii) and iii).

It was another object of the invention to provide membranes that are less prone to fouling.

This objective has been achieved by a membrane, comprising a polymer comprising at least one oxazoline according to formula

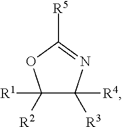

wherein R¹, R², R³ and R⁴ independently denote a hydrogen atom, a halogen atom, an alkyl group, an aralkyl group, a phenyl group, or a substituted phenyl group, and R⁵ denotes a noncyclic organic group having an unsaturated bond reactive in radical polymerization.

The concept of a membrane is generally known in the art. In the context of this application a membrane shall be understood to be a thin, semipermeable structure capable of separating two fluids or separating molecular and/or ionic components or particles from a liquid. A membrane acts as a selective barrier, allowing some particles, substances or chemicals to pass through while retaining others.

Membranes according to the invention can for example be microporous (average pore diameter smaller than 2 nm), mesoporous (average pore diameter from 2 nm to 50 nm) or macroporous (average pore diameter above 50 nm). Average pore diameters in this context are determined according to DIN 14652:2007-09 through correlation with the molecular weight cutoff of a membrane.

Suitable membranes or the separation layer of suitable membranes can be made of at least one inorganic material like a ceramic or at least one organic polymer.

Examples of inorganic materials are clays, silicates, silicon carbide, aluminium oxide, zirconium oxide or graphite. Such membranes made of inorganic materials are normally made by applying pressure or by sintering of finely ground powder. Membranes made of inorganic materials may be composite membranes comprising two, three or more layers.

In one embodiment, membranes made from inorganic materials comprise a macroporous support layer, optionally an intermediate layer and a separation layer.

In one aspect, this invention is directed to novel polymers comprising
i) at least one oxazoline according to formula

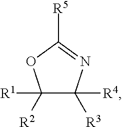

wherein R¹, R², R³ and R⁴ independently denote a hydrogen atom, a halogen atom, an alkyl group, an aralkyl group, a phenyl group, or a substituted phenyl group, and R⁵ denotes a noncyclic organic group having an unsaturated bond reactive in radical polymerization,
ii) optionally at least one antiadhesive monomer,
iii) optionally at least one biocidal monomer,
iv) optionally at least one further monomer,
   wherein said polymer comprises at least one monomer selected from monomers ii) and iii).

Preferably, said at least one oxazoline is selected from 2-Isopropenyl-2-oxazolin, 2-vinyl-2-oxazoline, 2-vinyl-4-methyl-2-oxazoline, 2-vinyl-5-methyl-2-oxazoline, 2-isopropenyl-4-methyl-2-oxazoline, 2-isopropenyl-5-methyl-2-oxazoline, 2-isopropenyl-4-ethyl-2-oxazoline and 2-isopropenyl-5-ethyl-2-oxazoline. Most preferably said at least one oxazoline is 2-Isopropenyl-2-oxazolin.

Polymers according to the invention normally comprise from 2 to 95% by weight of at least one oxazoline, preferably from 10 to 90% by weight. In one embodiment Polymers according to the invention comprise 20 to 50% by weight or 20 to 30% by weight. In another embodiment copolymers according to the invention comprise 60 to 85 or 70 to 80% by weight of at least one oxazoline.

Polymers according to the invention normally have a number average molecular weight of 3000 to 1000000, preferably 5000 to 300000, more preferably 10000 to 40000.

A “monomer”, for example “biocidal monomers”, “antiadhesive monomers” or “radically polymerizable monomers”, in this application shall, depending on the context, refer to such monomer in unpolymerized (monomeric) form or in polymerized form. When the term “monomer” is for example used in the context of a formulation or a composition, it normally refers to the unpolymerized form. When the term “monomer” is for example used in the context of a polymer or a coating, it normally refers to the polymerized form, in which said monomer is comprised in the polymer or coating.

Herein, “biocidal monomers” and “antiadhesive monomers” are sometimes referred to as “flux enhancing monomers”.

An antiadhesive monomer in the context of this application shall mean a monomer that imparts antiadhesive properties to the coating, be it by itself or in combination with other components. Antiadhesive properties or antiadhesive coating means that for example particles or biological material or biological organisms or degradation products of biological material or biological organisms have a lower tendency to adhere to the surface of a membrane having such antiadhesive properties. The degree of fouling and in particular biofouling of a membrane is thus reduced.

Antiadhesive coatings are sometimes also referred to as anti-sticking coatings, ‘stealth’ coatings or biopassive coatings.

The concept of antiadhesive polymers and coatings is for example disclosed in the following pieces of literature, which are incorporated herein by reference:
R. Konradi et al. Macromol. Rapid Commun. 2012, 33, 1663-1676;
R. G. Chapman, E. Ostuni, M. N. Liang, G. Meluleni, E. Kim, L. Yan, G. Pier, H. S. Warren, G. M. Whitesides, Langmuir 2001, 17, 1225;
R. G. Chapman, E. Ostuni, S. Takayama, R. E. Holmlin, L. Yan, G. M. Whitesides, J. Am. Chem. Soc. 2000, 122, 8303;
E. Ostuni, R. G. Chapman, R. E. Holmlin, S. Takayama, G. M. Whitesides, Langmuir 2001, 17, 5605;
E. Ostuni, R. G. Chapman, M. N. Liang, G. Meluleni, G. Pier, D. E. Ingber, G. M. Whitesides, Langmuir 2001, 17, 6336.

In one embodiment of the invention, suitable antiadhesive monomers are those, whose polymerization leads to the formation of antiadhesive coatings that are characterized by the presence of hydrophilic groups and preferentially the presence of hydrogen-bond-accepting groups, preferentially the absence of hydrogen-bond donating groups and preferentially the absence of net charge.

Suitable antiadhesive monomers are for example selected from
a) esters of (meth)acrylic acid with polyols
b) vinyl ethers of polyols
c) hydrophilic macromonomers different from monomers a) and b)
d) N-vinyl compounds
e) Low molecular weight hydrophilic (meth)acrylamides
f) (meth)acrylates or (meth)acrylamides bearing epoxy groups
g) monomers having a betain structure
h) hydrophilic monomers different from those mentioned under a) to g).
i) Ion pair comonomers

Suitable esters of (meth)acrylic acid with polyols a) are preferably esters with polyols that are hydrophilic and with which coatings can be prepared that show antiadhesive properties as described above.

In one embodiment, suitable esters of (meth)acrylic acid with polyols are polyols, in which each OH group is esterified with (meth)acrylic acid.

In one embodiment, suitable esters of (meth)acrylic acid with polyols are polyols, in which at least one OH group is esterified with (meth)acrylic acid and at least one OH group is not esterified.

In one embodiment, suitable esters of (meth)acrylic acid with polyols are polyols, in which at least one OH group is esterified with (meth)acrylic acid and at least one OH group is etherified with an alcohol like methanol, ethanol, propanol or a polyol like ethyleneglycol, neopentylglycol, trimethylolpropane, glycerol, trimethylolethane, pentaerythritol or dipentaerythritol, (poly)saccharide, in particular sorbitol.

Examples of suitable esters of (meth)acrylic acid with polyols are for example (meth)acrylates of alkoxylated polyols like ethyleneglycol, neopentylglycol, trimethylolpropane, glycerol, trimethylolethane, pentaerythritol, dipentaerythritol, or (poly)saccharide, in particular sorbitol bearing 1 to 100, preferably 1 to 50 ethoxy, propoxy, mixed ethoxy and propoxy, more preferably exclusively ethoxy groups per OH-group of the polyol.

More Preferably, suitable esters of (meth)acrylic acid with polyols are (meth)acrylates of, with respect to each OH group of the polyol, singly to hundred-fold, more preferably triply to 50-fold, in particular triply to vigintuply (20-fold) ethoxylated, propoxylated or mixedly ethoxylated and propoxylated, and more particularly exclusively ethoxylated, neopentylglycol, trimethylolpropane, glycerol, trimethylolethane, pentaerythritol, dipentaerythritol, or (poly)saccharide, in particular sorbitol.

Particularly preferred esters of (meth)acrylic acid with polyols are
ethylene glycol di(meth)acrylate, diethylene glycol di(meth)acrylate, triethylene glycol di(meth)acrylate, oligoethylene glycol di(meth)acrylate, polyethylene glycol di(meth)acrylate,
ethylene glycol mono(meth)acrylate, diethylene glycol mono(meth)acrylate, triethylene glycol mono(meth)acrylate, oligoethylene glycol mono(meth)acrylate, polyethylene glycol mono(meth)acrylate,
Ethylene glycol methyl ether(meth)acrylate, di(ethylene glycol) methyl ether(meth)acrylate, tri(ethylene glycol) methyl ether(meth)acrylate, oligo(ethylene glycol) methyl ether(meth)acrylate, poly(ethylene glycol) methyl ether(meth)acrylate,
glycerol tri(meth)acrylate, glycerol alkoxylate tri(meth)acrylate, preferentially glycerol ethoxylate tri(meth)acrylate
trimethylolpropane tri(meth)acrylate, trimethylolpropane alkoxylate tri(meth)acrylate, preferentially trimethylolpropane ethoxylate tri(meth)acrylate
pentaerythritol tetra(meth)acrylate, pentaerythritol alkoxylate tetra(meth)acrylate, preferentially pentaerythritol ethoxylate tetra(meth)acrylate
pentaerythritol tri(meth)acrylate, pentaerythritol alkoxylate tri(meth)acrylate, preferentially pentaerythritol ethoxylate tri(meth)acrylate
dipentaerythritol penta(meth)acrylate, dipentaerythritol alkoxylate penta(meth)acrylate, preferentially dipentaerythritol ethoxylate penta(meth)acrylate
sorbitol hexa(meth)acrylate, sorbitol alkoxylate hexa(meth)acrylate, preferentially sorbitol ethoxylate hexa(meth)acrylate; sorbitol penta(meth)acrylate, sorbitol alkoxylate penta(meth)acrylate, preferentially sorbitol ethoxylate penta(meth)acrylate; sorbitol tetra(meth)acrylate, sorbitol alkoxylate tetra(meth)acrylate, preferentially sorbitol ethoxylate tetra(meth)acrylate; sorbitol tri(meth)acrylate, sorbitol alkoxylate tri(meth)acrylate, preferentially sorbitol ethoxylate tri(meth)acrylate

In one embodiment, suitable esters of (meth)acrylic acid with polyols do not include (meth)acrylic esters with polyalkyleneoxides like polyethyleneoxides.

In another embodiment, suitable esters of (meth)acrylic acid with polyols do not include esters of (meth)acrylic aid with polyvalent alcohols or phenols.

Suitable antiadhesive monomers b) are vinyl ethers of polyols or vinyl ethers of alkoxylated polyols.

Suitable vinyl ethers of polyols are preferably ethers with that are hydrophilic and with which coatings can be prepared that show antiadhesive properties as described above.

In one embodiment, suitable vinyl ethers of polyols are polyols, in which each OH group is etherified vinyl alcohol.

In one embodiment, suitable vinyl ethers of polyols are polyols, in which at least one OH group is etherified with vinyl alcohol and at least one OH group is not etherified.

In one embodiment, suitable vinyl ethers of polyols are polyols, in which at least one OH group is etherified vinylalcohol and at least one OH group is etherified with a saturated alcohol like methanol, ethanol, propanol or a polyol like ethyleneglycol, neopentylglycol, trimethylolpropane, glycerol, trimethylolethane, pentaerythritol, dipentaerythritol, (poly)saccharide like sorbitol.

Examples of suitable vinyl ethers of polyols are for example vinyl ethers of alkoxylated polyols like ethyleneglycol, neopentylglycol, trimethylolpropane, glycerol, trimethylolethane, pentaerythritol or dipentaerythritol bearing 1 to 100, preferably 1 to 50 ethoxy, propoxy, mixed ethoxy and propoxy, more preferably exclusively ethoxy groups per OH-group of the polyol.

Preferred vinyl ethers of polyols are ethylene glycol divinylether, diethylene glycol divinylether, triethylene glycol divinylether, oligoethylene glycol divinylether, polyethylene glycol divinyl ether, methoxyethylene glycol monovinylether, methoxy diethylene glycol monovinylether, methoxy triethylene glycol monovinylether, methoxy oligoethylene glycol monovinylether, methoxy polyethylene glycol monovinyl ether.

Suitable antiadhesive monomers c) are hydrophilic macromonomers such as (meth)acryloyl-, (meth)acrylamide- and vinylether-modified hydrophilic polymers, preferentially (meth)acryloyl-modified polyvinyl alcohol, (meth)acryloyl-modified partially hydrolyzed polyvinyl acetate, (meth)acryloyl-modified poly(2-alkyl-2-oxazoline), (meth)acrylamide-modified poly(2-alkyl-2-oxazoline), in particular (meth)acryloyl and (meth)acrylamide-modified poly(2-methyl-2-oxazoline) and (meth)acryloyl- and (meth)acrylamide-modified poly(2-ethyl-2-oxazoline), (meth)acryloyl- and (meth)acrylamide-modified poly(vinyl pyrrolidone), (meth)acryloyl- and (meth)acrylamide-modified hydrophilic polypeptoids, (meth)acryloyl- and (meth)acrylamide-modified polyphosphorylcholine, (meth)acryloyl- and (meth)acrylamide-modified polysulfobetain, (meth)acryloyl- and (meth)acrylamide-modified polycarbobetain, (meth)acryloyl- and (meth)acrylamide-modified polyampholyte.

Suitable antiadhesive monomers d) are N-vinyl compounds such as N-vinyl pyrrolidone, N-vinylCaprolactam, N-vinylcaprolactone or N-vinyl-2-piperidone.

Suitable antiadhesive monomers e) are low molecular weight (meth)acrylamides with a molecular weight below 200, preferably below 150.

Preferred low molecular weight (meth)acrylamides are those according to formula

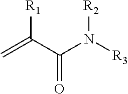

with R₁═H or CH₃, R₂, R₃=independently from each other H, methyl, ethyl, 1-propyl, 2-propyl, 1-butyl, 2-butyl.

Preferred alkylated (meth)acrylamides are: R₂═R₃═H (=(meth)acrylamide), R₂═R₃=methyl (═N,N-dimethyl(meth)acrylamide), R₂═R₃=ethyl (═N,N-diethyl(meth)acrylamide), R₂═H, R₃₌₂-propyl (═N-isopropyl(meth)acrylamide).

Suitable (meth)acrylates or (meth)acrylamides bearing epoxy groups f) are for example glycidyl(meth)acrylate.

Suitable monomers having a betain structure g) are for example sulfobetaines or carbobetaines of (meth)acrylates or (meth)acrylamides, sulfonyl- or carboxy-modified vinylimidazolium betains, sulfonyl- or carboxy-modified vinylpyridinium betains, sulfobetain- or carbobetain-modified styrenyls, phosphobetain(meth)acrylates or Phosphobetain(meth)acrylamides.

Suitable sulfobetaines or carbobetaines of (meth)acrylates or (meth)acrylamides are for example sulfobetain(meth)acrylates, sulfobetain(meth)acrylamides, carbobetain(meth)acrylates, carbobetain(meth)acrylamides of general formula

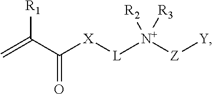

wherein
R₁═H, Methyl;
R₂, R₃=alkyl, aryl, aralkyl, preferentially R₂═R₃=Methyl;
X═O, NH;
L=alkyl, aryl, aralkyl. L may contain heteroatoms in particular one or several groups of (CH₂)ₙO, (CH₂)ₙNH, n is preferentially 2-3; preferably L is methylene, ethylene or propylene; in particular ethylene or propylene.
Z=alkyl, aryl, aralkyl. Z may contain heteroatoms in one or several groups of (CH₂)ₙO, (CH₂)ₙNH, n is preferentially 2-3; preferably Z is methylene, ethylene, propylene, butylene
Y=sulfonate or carboxylate

Examples of suitable sulfobetaines or carbobetaines of (meth)acrylates or (meth)acrylamides are:

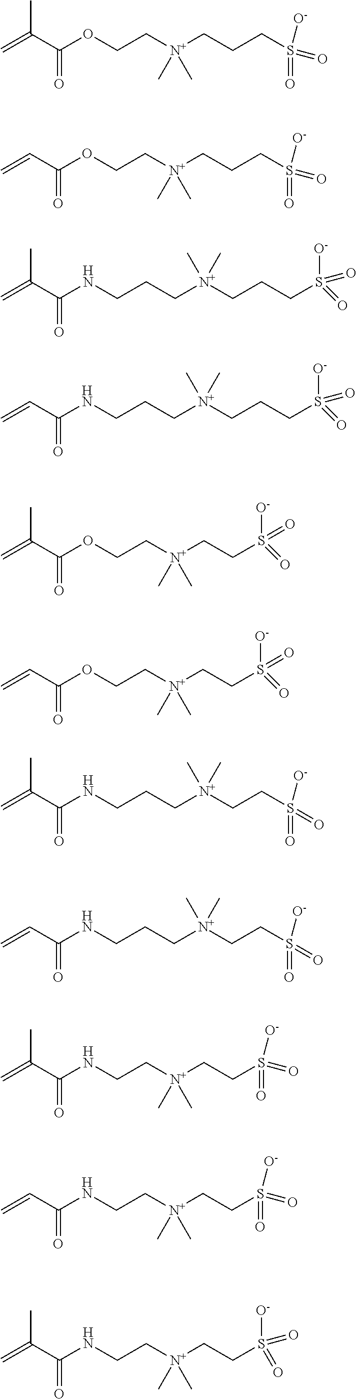

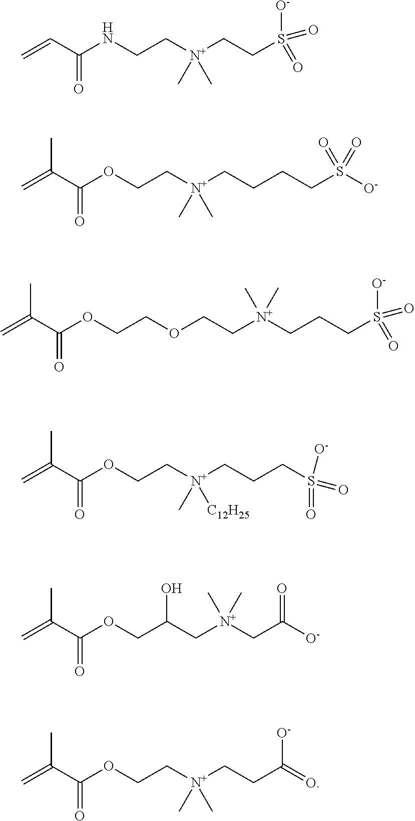

Further suitable sulfobetaines or carbobetaines of (meth)acrylates or (meth)acrylamides are sulfobetain di(meth)acrylates, sulfobetain di(meth)acrylamides, carbobetain di(meth)acrylates and carbobetain di(meth)acrylamides. Preferred sulfobetaines or carbobetaines of (meth)acrylates or (meth)acrylamides are of the general formula

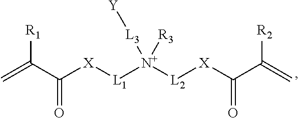

wherein
R₁, R₂═H, Methyl
R₃=alkyl, aryl, aralkyl, preferably R₃=Methyl
X═O, NH
L₁, L₂=independently from each other alkyl, aryl, aralkyl. L may contain heteroatoms in particular one or several groups of (CH₂)ₙO, (CH₂)ₙNH, n is preferentially 2-3; preferably L=methylene, ethylene, propylene; in particular ethylene and propylene;
L₃=alkyl, aryl, aralkyl. L₃ may contain heteroatoms in particular one or several groups of (CH₂)ₙO, (CH₂)ₙNH, n is preferably 2-3; preferably L₃=methylene, ethylene, propylene, butylene;
Y=sulfonate or carboxylate.

Further examples of suitable sulfobetaines or carbobetaines of (meth)acrylates or (meth)acrylamides are

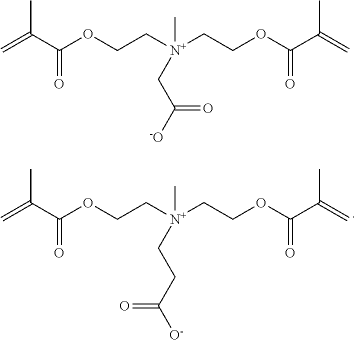

Suitable sulfonyl- or carboxy-modified vinylimidazolium betains are for example sulfonylor carboxy-modified vinylimidazolium betains of general formula

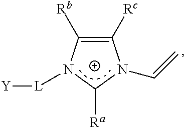

wherein
R^{a}, R^{b} and R^{c} independently of one another are an H atom or an organic radical having up to 22 C atoms, preferably R^{a}═R^{b}═R^{c}═H,
L=alkyl, aryl, aralkyl. L may contain heteroatoms in particular one or several groups of (CH₂)ₙO, (CH₂)ₙNH, n is preferentially 2-3; preferably L=methylene, ethylene, propylene, butylene,
Y=sulfonate or carboxylate.

Examples of sulfonyl- or carboxy-modified vinylimidazolium betains are:

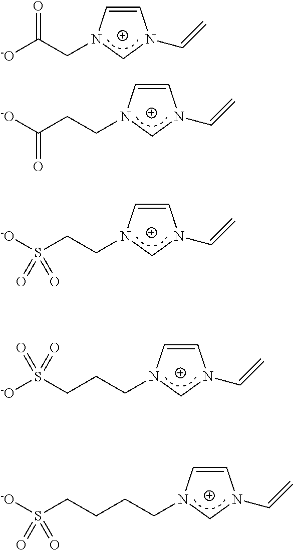

Suitable sulfonyl- or carboxy-modified vinylpyridinium betains are for example those according to the general formula

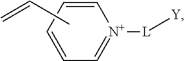

wherein
L=alkyl, aryl, aralkyl; L may contain heteroatoms in particular one or several groups of (CH₂)ₙO, (CH₂)ₙNH, n is preferably 2-3;
   preferably L=methylene, ethylene, propylene, butylene;
Y=sulfonate or carboxylate.

Examples of sulfonyl- or carboxy-modified vinylpyridinium betains include

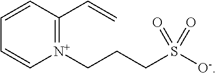

Suitable Sulfobetain- or Carbobetain-modified styrenyls are for example those according to the general formula

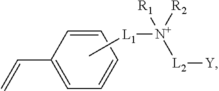

wherein
R₁, R₂=alkyl, aryl, aralkyl, preferably R₁═R₂=Methyl,
L₁, L₂=independently from each other alkyl, aryl, aralkyl; L may contain heteroatoms in particular one or several groups of (CH₂)ₙO, (CH₂)ₙNH, n is preferentially 2-3; preferably L=methylene, ethylene, propylene, butylene; in particular ethylene and propylene,
Y=sulfonate or carboxylate.

Examples of Sulfobetain- or Carbobetain-modified styrenyls include:

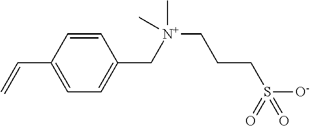 [SMILES: C=Cc1ccc(C[N+](C)(C)CCCS(=O)(=O)[O-])cc1]

Suitable phosphobetain(meth)acrylates or phosphobetain(meth)acrylamides are those of the general formula

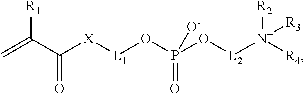

wherein
R₁═H, Methyl,
R₂, R₃, R₄=alkyl, aryl, aralkyl, preferentially R₂═R₃═R₃=Methyl,
X═O, NH,
L₁, L₂=independently from each other alkyl, aryl, aralkyl. L₁, L₂ may independently from each other contain heteroatoms in particular one or several groups of (CH₂)ₙO, (CH₂)ₙNH, n is preferably 2-3; preferably L₁, L₂ are independently from each other methylene, ethylene, propylene, butylene; in particular and independently from each other ethylene and propylene.

Examples of phosphobetain(meth)acrylates or phosphobetain(meth)acrylamides include

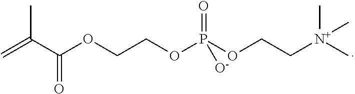 [SMILES: C=C(C)C(=O)OCCOP(=O)([O-])OCC[N+](C)(C)C]

Suitable hydrophilic monomers h) different from those mentioned above are hydroxyethyl(meth)acrylate, Vinyl alcohol, (Meth)acryloyl and (meth)acrylamide-modified mono- and oligosaccharides.

Suitable Ion pair comonomers i) are in particular ion pairs of ammonium-modified (meth)acrylates or (meth)acrylamides and sulfo-, carboxy-, phosphonyl or phosphoryl-modified (meth)acrylates or (meth)acrylamides. A preferred example is the combination

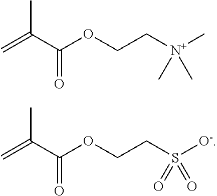

In one embodiment of the invention polymers according to the invention comprise only one antiadhesive monomer.

In one embodiment of the invention polymers according to the invention comprise two or more antiadhesive monomers.

A biocidal monomer in the context of this application shall mean a monomer that imparts biocidal properties to the coating, be it by itself or in combination with other components. Biocidal properties or biocidal coating means that living biological organisms like plants, algae, bacteria, cyanobacteria, fungi, yeasts, molds, protozoa, viruses, mycoplasma, other microorganisms or higher organisms such as barnacles are deterred, controlled and/or inactivated by said coating. The degree of fouling and in particular biofouling of a membrane is thus reduced.

The mechanisms of such biocidal effects are not entirely understood. It is assumed the biocidal effect of biocidal monomers or coatings can for example be due to the interfering with the production of the bacterial plasma wall, interfering with protein synthesis, nucleic acid synthesis, or plasma membrane integrity, or to inhibiting critical biosynthetic pathways in the bacteria.

Suitable biocidal monomers are for example selected from
j) vinyl-imidazolium compounds
k)ethylenically unsaturated monomers bearing quarternary ammonium or phosphonium groups
l) diallyldialkylammoniumchlorides
m) alkylaminoalkyl(meth)acrylates and alkylaminoalkyl(meth)acrylamides
n) Polylysine(meth)acrylamides or (meth)acrylates
o) alkyl-4-vinylpridinium and alkyl-2-vinyl-pyridinium salts, in particular bromides and iodides
p) ethylenically unsaturated monomers bearing guanide and biguanide groups
q) halamines

Further biocidal monomers and corresponding polymers can be found for example in Tatsuo Tashiro Macromol. Mater. Eng. 2001, 286, 63-87.

Suitable vinyl-imidazolium compounds j) are in particular 3-vinyl-imidazol-1-ium compounds. These are preferably selected from a 3-vinyl-imidazol-1-ium compounds having the formula (III)

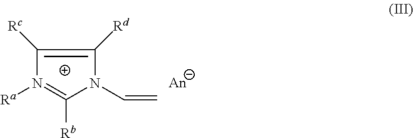

in which R^{a} is an organic radical having 1 to 22 C atoms,
R^{b}, R^{c} and R^{d} independently of one another are an H atom or an organic radical having up to 22 C atoms and An⁻ is an anion.

R^{a} is an organic radical having 1 to 22 C atoms. The organic radical may also comprise further heteroatoms, more particularly oxygen atoms, nitrogen, sulfur or phosphorus atoms, or functionnal groups, as for example hydroxyl groups, ether groups, ester groups, or carbonyl groups.

More particularly R^{a} is a hydrocarbon radical which apart from carbon and hydrogen may further comprise at most hydroxyl groups, ether groups, ester groups or carbonyl groups.

R^{a} with particular preference is a hydrocarbon radical having 1 to 22 C atoms, more particularly having 4 to 20 C atoms, which comprises no other heteroatoms, e.g., oxygen or nitrogen. The hydrocarbon radical may be aliphatic (in which case unsaturated aliphatic groups are also included, but less preferred) or aromatic, or may comprise both aromatic and aliphatic groups. Preferably R^{a} is an aliphatic hydrocarbon radical.

Examples of hydrocarbon radicals include the phenyl group, benzyl group, a benzyl group or phenyl group substituted by one or more C₁ to C₄ alkyl groups, or the mesityl group, alkyl groups and alkenyl groups, more particularly the alkyl group.

With very particular preference R^{a} is a C₄ to C₂₂ alkyl group, preferably a C₄ to C₁₈.

Examples for R^{a} are methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, heptyl, octyl, nonyl, decyl, undecyl, dodecyl, tridecyl, tetradecyl, pentadecyl, hexadecyl, heptadecyl, octadecyl, nonadecyl, icosyl, phenylmethyl(benzyl), diphenylmethyl, triphenylmethyl, 2-phenylethyl, 3-phenylpropyl, cyclopentylmethyl, 2-cyclopentylethyl, 3-cyclopentylpropyl, cyclohexylmethyl, 2-cyclohexylethyl, and 3-cyclohexylpropyl.

With very particular preference R^{a} is a 1-butyl, 2-butyl, 2-methyl-1-propyl (isobutyl), 2-methyl-2-propyl (tert-butyl), 1-pentyl, 2-pentyl, 3-pentyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, heptyl, octyl, nonyl, decyl, undecyl, dodecyl, tridecyl, tetradecyl, pentadecyl, hexadecyl, heptadecyl, octadecyl, nonadecyl, or icosyl group, with the butyl, pentyl, hexyl, octyl, decyl, dodecyl, tetradecyl, hexadecyl, and octadecyl groups having particular importance.

In one preferred embodiment R^{b} is an H atom.

In another preferred embodiment R^{b} is an alkyl group, as for example a C₁ to C₁₈ alkyl group, preferably a C₁ to C₁₆, more preferably a C₁ to C₁₄, very preferably C₁ to C₁₂, and more particularly C₁ to C₁₀ alkyl group. For the radical R^{b}, a C₁ to C₆ alkyl group represents one particular embodiment, and in a very particular embodiment the alkyl group is a C₁ to C₄ alkyl group.

R^{c} and R^{d} are preferably independently of one another a hydrogen atom or an organic radical having 1 to 10 C atoms. The organic radical may also comprise further heteroatoms, more particularly oxygen atoms, nitrogen, sulfur or phosphorus atoms, or functional groups, as for example hydroxyl groups, ether groups, ester groups, or carbonyl groups.

More particularly R^{c} and R^{d} are a hydrocarbon radical which apart from carbon and hydrogen may further comprise at most hydroxyl groups, ether groups, ester groups or carbonyl groups.

R^{c} and R^{d} with particular preference are independently of one another a hydrocarbon radical having 1 to 20 C atoms, more particularly having 1 to 10 C atoms, which comprises no other heteroatoms, e.g., oxygen or nitrogen. The hydrocarbon radical may be aliphatic (in which case unsaturated aliphatic groups are also included) or aromatic, or may comprise both aromatic and aliphatic groups.

Examples of hydrocarbon radicals include the phenyl group, benzyl group, a benzyl group or phenyl group substituted by one or more C₁ to C₄ alkyl groups, or the mesityl group, alkyl groups and alkenyl groups, more particularly the alkyl group.

With very particular preference R^{c} and R^{d} are a hydrogen atom or a C₁ to C₁₀ alkyl group. A partitularly preferred alkyl group is a C₁ to C₆ alkyl group, and in one particular embodiment the alkyl group is a C₁ to C₄ alkyl group.

With very particular preference R^{c} and R^{d} are independently of one another a methyl, ethyl, n-propyl, isopropyl, n-butyl, sec-butyl or tert-butyl group, with the methyl, ethyl, n-propyl, and n-butyl groups having particular importance.

In one particular embodiment R^{c} and R^{d} are each H atoms.

In a very particular embodiment R^{b}, R^{c}, and R^{d} are each H atoms.

Examples of imidazolium ions are

1-butyl-3-vinyl-imidazol-1-ium, 1-pentyl-3-vinyl-imidazol-1-ium, 1-hexyl-3-vinyl-imidazol-1-ium, 1-octyl-3-vinyl-imidazol-1-ium, 1-decyl-3-vinyl-imidazol-1-ium, 1-dodecyl-3-vinyl-imidazol-1-ium, 1-tetradecyl-3-vinyl-imidazol-1-ium, 1-hexadecyl-3-vinyl-imidazol-1-ium, 1-octadecyl-3-vinyl-imidazol-1-ium, 1-hexyl-2-methyl-3-vinyl-imidazol-1-ium, 1-octyl-2-methyl-3-vinyl-imidazol-1-ium, 1-decyl-2-methyl-3-vinyl-imidazol-1-ium, 1-dodecyl-2-methyl-3-vinyl-imidazol-1-ium, 1-tetradecyl-2-methyl-3-vinyl-imidazol-1-ium, 1-hexadecyl-2-methyl-3-vinyl-imidazol-1-ium, and 1-octadecyl-2-methyl-3-vinyl-imidazol-1-ium.

Preferred imidazolium ions are 1-butyl-3-vinyl-imidazol-1-ium, 1-hexyl-3-vinyl-imidazol-1-ium, 1-octyl-3-vinyl-imidazol-1-ium, 1-decyl-3-vinyl-imidazol-1-ium, 1-dodecyl-3-vinyl-imidazol-1-ium, 1-tetradecyl-3-vinyl-imidazol-1-ium, 1-hexadecyl-3-vinyl-imidazol-1-ium, and 1-octadecyl-3-vinyl-imidazol-1-ium.

The anion An⁻ is any desired anion, preferably a halide or carboxylate anion, preferably a halide anion.

Anions other than carboxylate anion are described, for example, in WO 2007/090755, particularly from page 20 line 36 to page 24 line 37 therein, which is hereby made part of the present disclosure content by reference.

Suitable anions are more particularly those from

the group of the halides and halogen-containing compounds of the following formulae: F⁻, Cl⁻, Br⁻, I⁻, BF₄⁻, PF₆⁻, AlCl₄⁻, Al₂Cl₇⁻, Al₃Cl₁₀⁻, AlBr₄⁻, FeCl₄⁻, BCl₄⁻, SbF₆⁻, AsF₆⁻, ZnCl₃⁻, SnCl₃⁻, CuCl₂⁻, CF₃SO₃⁻, (CF₃SO₃⁻)₂N⁻, CF₃CO₂⁻, CCl₃CO₂⁻, CN⁻, SCN⁻, OCN⁻, NO²⁻, NO³⁻, N(CN)⁻, N₃⁻;
the group of the sulfates, sulfites, and sulfonates, of the following general formulae:
SO₄²⁻, HSO₄⁻, SO₃²⁻, HSO₃⁻, R^{e}OSO₃⁻, R^{e}SO₃⁻;
the group of the phosphates, of the following general formulae:
PO₄³⁻, HPO₄²⁻, H₂PO₄⁻, R^{e}PO₄²⁻, HR^{e}PO₄⁻, R^{e}R^{f}PO₄⁻;
the group of the phosphonates and phosphinates, of the following general formula:

### R^{e}HPO₃⁻, R^{e}R^{f}PO₂⁻, R^{e}R^{f}PO₃⁻;

the group of the phosphites, of the following general formulae:
PO₃³⁻, HPO₃²⁻, H₂PO₃⁻, R^{e}PO₃²⁻, R^{e}HPO₃⁻, R^{e}R^{f}PO₃⁻;
the group of the phosphonites and phosphinites, of the following general formula:
R^{e}R^{f}PO₂⁻, R^{e}HPO₂⁻, R^{e}R^{f}PO⁻, R^{e}HPO⁻;
the group of the borates, of the following general formulae:
BO₃³⁻, HBO₃²⁻, H₂BO₃⁻, R^{e}R^{f}BO₃⁻, R^{e}HBO₃⁻, R^{e}BO₃²⁻, B(OR^{e})(OR^{f})(OR^{g})(OR^{h})⁻, B(HSO₄)⁻, B(R^{e}SO₄)⁻;
the group of the boronates, of the following general formulae:

### R^{e}BO₂²⁻, R^{e}R^{f}BO⁻;

the group of the carbonates and carbonic esters, of the following general formulae:

### HCO₃⁻, CO₃²⁻, R^{e}CO₃⁻;

the group of the silicates and silicic acid esters, of the following general formulae:
SiO₄⁴⁻, HSiO₄³⁻, H₂SiO₄²⁻, H₃SiO₄⁻, R^{e}SiO₄³⁻, R^{e}R^{f}SiO₄²⁻, R^{e}R^{f}R^{g}SiO₄⁻, HR^{e}SiO₄²⁻, H₂R^{e}SiO₄⁻, HR^{e}R^{f}SiO₄⁻;
the group of the alkyl silane and aryl silane salts, of the following general formulae:
R^{e}SiO₃³⁻, R^{e}R^{f}SiO₂²⁻, R^{e}R^{f}R^{g}SiO⁻, R^{e}R^{f}R^{g}SiO₃⁻, R^{e}R^{f}R^{g}SiO₂⁻, R^{e}R^{f}SiO₃²⁻;
the group of the carboximides, bis(sulfonyl)imides, and sulfonylimides, of the following general formulae:

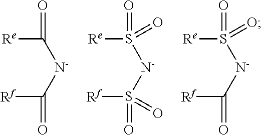

the group of the methides, of the following general formula:

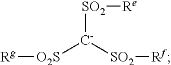

the group of the alkoxides and aryl oxides, of the following general formulae:

### R^{e}O⁻;

the group of the halometallates, of the following general formula:
[MᵣHalₜ]^{s-},
where M is a metal and Hal is fluorine, chlorine, bromine or iodine, r and t are positive integers, and indicate the stoichiometry of the complex, and s is a positive integer and indicates the charge of the complex;
the group of the sulfides, hydrogen sulfides, polysulfides, hydrogenpolysulfides, and thiolates, of the following general formulae:

### S²⁻, HS⁻, [Sᵥ]²⁻, [HSᵥ]⁻, [R^{e}S]⁻,

where visa positive integer from 2 to 10; and
the group of the complex metal ions such as Fe(CN)₆³⁻, Fe(CN)₆⁴⁻, MnO₄⁻, Fe(CO)₄⁻.

In the above anions, R^{e}, R^{f}, R^{g}, and R^{h} independently of one another are in each case hydrogen;

C₁-C₃₀ alkyl and its aryl-, heteroaryl-, cycloalkyl-, halogen-, hydroxyl-, amino-, carboxyl-, formyl-, —O—, —CO—, —CO—O— or —CO—N< substituted components, such as, for example, methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, heptyl, octyl, nonyl, decyl, undecyl, dodecyl, tridecyl, tetradecyl, pentadecyl, hexadecyl, heptadecyl, octadecyl, nonadecyl, icosyl, henicosyl, docosyl, tricosyl, tetracosyl, pentacosyl, hexacosyl, heptacosyl, octacosyl, nonacosyl, triacontyl, phenylmethyl (benzyl), diphenylmethyl, triphenylmethyl, 2-phenylethyl, 3-phenylpropyl, cyclopentylmethyl, 2-cyclopentylethyl, 3-cyclopentylpropyl, cyclohexylmethyl, 2-cyclohexylethyl, 3-cyclohexylpropyl, methoxy, ethoxy, formyl, acetyl or C_{q}F_{2(q-a)+(1-b)}H_{2a+b} with q≦30, 0≦a≦q and b=0 or 1 (for example, CF₃, C₂F₅, CH₂CH₂—C_{(q-2)}F_{2(q-2)+1}, C₆F₁₃, C₈F₁₇, C₁₀F₂₁, C₁₂F₂₅);
C₃-C₁₂ cycloalkyl and its aryl-, heteroaryl-, cycloalkyl-, halogen-, hydroxyl-, amino-, carboxyl-, formyl-, —O—, —CO— or —CO—O-substituted components, such as, for example, cyclopentyl, 2-methyl-1-cyclopentyl, 3-methyl-1-cyclopentyl, cyclohexyl, 2-methyl-1-cyclohexyl, 3-methyl-1-cyclohexyl, 4-methyl-1-cyclohexyl or C_{q}F_{2(q-a)-(1-b)}H_{2a-b} with q≦30, 0≦a≦q and b=0 or 1;
C₂-C₃₀ alkenyl and its aryl-, heteroaryl-, cycloalkyl-, halogen-, hydroxyl-, amino-, carboxyl-, formyl-, —O—, —CO— or —CO—O-substituted components, such as, for example, 2-propenyl, 3-butenyl, cis-2-butenyl, trans-2-butenyl or C_{q}F_{2(q-a)-(1-b)}H_{2a-b} with q≦30, 0≦a≦q and b=0 or 1;
C₃-C₁₂ cycloalkenyl and its aryl-, heteroaryl-, cycloalkyl-, halogen-, hydroxyl-, amino-, carboxyl-, formyl-, —O—, —CO— or —CO—O-substituted components, such as, for example 3-cyclopentenyl, 2-cyclohexenyl, 3-cyclohexenyl, 2,5-cyclohexadienyl or C_{q}F_{2(q-a)-3(1-b)}H_{2a-3b} with q≦30, 0≦a≦q and b=0 or 1;
aryl or heteroaryl having 2 to 30 carbon atoms, and their alkyl-, aryl-, heteroaryl-, cycloalkyl-, halogen-, hydroxy-, amino-, carboxy-, formyl-, —O—, —CO— or —CO—O-substituted components, such as, for example, phenyl, 2-methylphenyl(2-tolyl), 3-methylphenyl(3-tolyl), 4-methylphenyl, 2-ethylphenyl, 3-ethylphenyl, 4-ethylphenyl, 2,3-dimethylphenyl, 2,4-dimethylphenyl, 2,5-dimethylphenyl, 2,6-dimethylphenyl, 3,4-dimethylphenyl, 3,5-dimethylphenyl, 4-phenylphenyl, 1-naphthyl, 2-naphthyl, 1-pyrrolyl, 2-pyrrolyl, 3-pyrrolyl, 2-pyridinyl, 3-pyridinyl, 4-pyridinyl or C₆F₍₅₋ₐ₎Hₐ with 0≦a≦5; or
two radicals denote an unsaturated, saturated or aromatic ring which is unsubstituted or substituted by functional groups, aryl, alkyl, aryloxy, alkyloxy, halogen, heteroatoms and/or heterocycles, and which is uninterrupted or interrupted by one or more oxygen and/or sulfur atoms and/or by one or more substituted or unsubstituted imino groups.

In the above anions, R^{e}, R^{f}, R^{g}, and R^{h} are preferably each independently of one another a hydrogen atom or a C₁ to C₁₂ alkyl group or a CF₃.

Examples of anions include chloride; bromide; iodide; thiocyanate; isothiocyanate; azide, hexafluorophosphate; trifluoromethanesulfonate; methanesulfonate; the carboxylates, especially formate; acetate; mandelate; carbonates, preferably methyl carbonate and n-butyl carbonate, nitrate; nitrite; trifluoroacetate; sulfate; hydrogensulfate; methylsulfate; ethylsulfate; 1-propylsulfate; 1-butylsulfate; 1-hexylsulfate; 1-octylsulfate; phosphate; dihydrogenphosphate; hydrogen-phosphate; C₁-C₄ dialkylphosphates; propionate; tetrachloroaluminate; Al₂Cl₇—; chlorozincate; chloroferrate; bis(trifluoromethylsulfonyl)imide; bis(pentafluoroethylsulfonyl)imide; bis(methylsulfonyl)imide; bis(p-tolylsulfonyl)imide; tris(trifluoromethylsulfonyl)methide; bis(pentafluoroethylsulfonyl)methide; p-tolylsulfonate; tetracarbonylcobaltate; dimethylene glycol monomethyl ether sulfate; oleate; stearate; acrylate; methacrylate; maleate; hydrogencitrate; vinylphosphonate; bis(pentafluoroethyl)phosphinate; borates such as bis[salicylato(2-)]borate, bis[oxalato(2-)]borate, bis[1,2-benzenediolato(2-)-O,O′]borate, tetracyanoborate, tetrafluoroborate; dicyanamide; tris(pentafluoroethyl)trifluorophosphate; tris(heptafluoropropyl)trifluorophosphate, cyclic arylphosphates such as pyrrocatechol-phosphate (C₆H₄O₂)P(O)O—, and chlorocobaltate.

Particularly preferred anions are those from the group of the halides, especially chloride, bromide, iodide, azide, thiocyanate, acetate, methyl carbonate, tetrafluoroborate, trifluoromethanesulfonate, methanesulfonate, bis(trifluoromethylsulfonyl)imide, ethylsulfate and diethyl phosphate.

Examples of suitable vinyl-imidazolium compounds j) include:

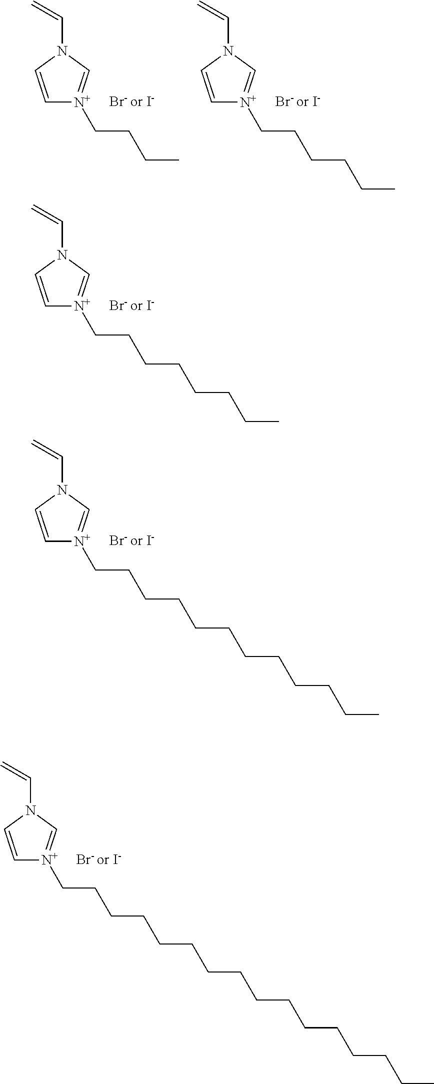

Suitable flux enhancing monomers bearing quarternary ammonium or phosphonium groups k) are for example selected from compounds of the general formula

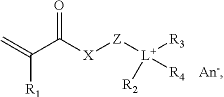

wherein
R1=H, methyl, preferably methyl,
X═O, NH preferably O,
Z=alkylene or polyoxyalkylene, preferably ethylene or polyoxyalkylene (polyalkylenglycol, preferably poly(ethylene glycol), poly(propylene glycol); poly(2-alkyl-2-oxazoline), preferably poly(2-methyl-2-oxazoline), poly(2-ethyl-2-oxazoline));
L=N, P; preferably N
R², R³, R⁴=alkyl; preferably R²═R³=methyl; R⁴=preferably C₆-C₂₂, more preferably C₈-C₁₈, especially preferably C₈-C₁₂, particularly preferably C₁₂;
An⁻: counterion, preferably bromide or iodide.

Examples of biocidal monomers bearing quarternary ammonium groups are for example

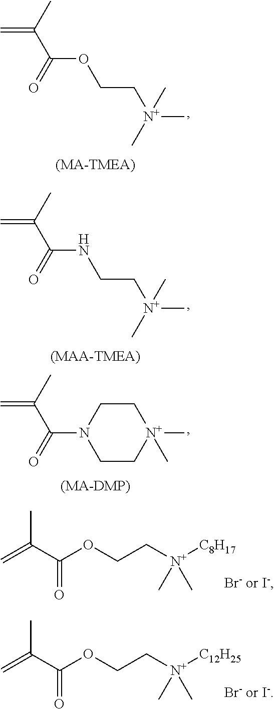

Further suitable flux enhancing monomers bearing quarternary ammonium groups are 3-methacryloyl aminopropyl-trimethyl ammoniumchloride, 2-methacryloyl oxyethyltrimethyl ammonium chloride, 2-Methacryloyloxyethyl-trimethylammoniummethosulfate, 3-acrylamidopropyl trimethylammoniumchloride, trimethylvinylbenzyl-ammoniumchlorid, 2-acryloyloxyethyl-4-benzoylbenzyl-dimethyl ammoniumbromide, 2-acryloyloxyethyltrimethylammoniummethosulfate, N,N,N-Trimethylammonium-ethylenebromide, 2-hydroxy N,N,N-trimethyl-3-[(2-methyl-1-oxo-2-propenyl)oxy]-ammoniumpropane chloride, N,N,N-Trimethyl-2-[(1-oxo-2-propenyl)oxy]-ammoniumethane-methylsulfate, N,N-Diethyl-N-methyl-2-[(1-oxo-2-propenyl)oxy]-ammoniumethane-methylsulfate, N,N,N-trimethyl-2-[(1-oxo-2-propenyl)oxy]-ammonium ethanechloride, N,N,N-trimethyl-2-[(2-methyl-1-oxo-2-propenyl)oxy]-ammonium ethanechloride, N,N,N-trimethyl-2-[(2-methyl-1-oxo-2-propenyl)oxy]-ammoniumethan-methylsulfate, N,N,N-triethyl-2-[(1-oxo-2-propenyl)amino]-ammoniumethan.

Further suitable biocidal monomers bearing quarternary ammonium or phosphonium groups are for example selected from compounds of the general formula

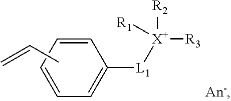

wherein
X═N, P; preferably N,
L₁=alkylene or polyoxyalkylene, preferably ethylene or polyoxyalkylene (polyalkylenglycol, preferably poly(ethylene glycol), poly(propylene glycol); poly(2-alkyl-2-oxazoline), preferably poly(2-methyl-2-oxazoline), poly(2-ethyl-2-oxazoline)),
R₁, R₂, R₃=independently alkyl, aryl or aralkyl;
   If X═N: preferably R₁═R₂=methyl; R₃=preferably C₆-C₂₂, more preferably C₈-C₁₈, especially preferably C₈-C₁₂, particularly preferably C₁₂;
   If X═P: preferably R₁═R₂═R₃=octyl, butyl or phenyl;
An⁻: counterion, preferably halide, most preferably chloride, bromide or iodide.

Examples of further suitable biocidal monomers bearing quarternary ammonium or phosphonium groups include:

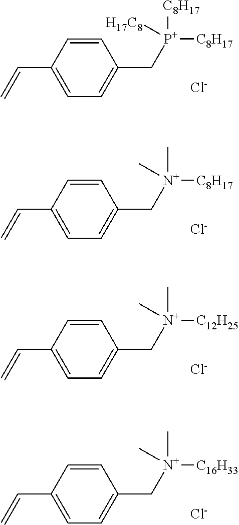

Suitable diallyldialkylammoniumchlorides I) are for example diallyldimethylammoniumchloride (DADMAC).

Suitable alkylaminoalkyl(meth)acrylate and alkylaminoalkyl(meth)acrylamide m) are for example those according to formula (I)

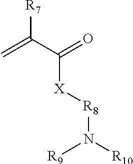

wherein
R₇ is H or CH₃,
R₈ is C₁-C₅ alkyl bi-radical,
R₉ and R₁₀ are independently H or C₁-C₅ alkyl radical which can be linear or branched,
and X is a divalent radical of —O—, —NH— or —NR₁₁, wherein R₁₁ is C₁-C₆ alkyl.

Preferred flux enhancing monomers according to formula (I) are 2-tert-butylaminoethyl(meth)acrylate (tBAEMA), 2-di methylaminoethyl(meth)acrylate, 2-diethylaminoethyl(meth)acrylate, 3-dimethylaminopropyl(meth)acrylate, N-3-dimethylaminopropyl(meth)acrylamide, and N-3-diethylaminopropyl(meth)acrylamide with the most preferred being 2-tert-butylaminoethyl(meth)acrylate (tBAEMA).

Suitable Polylysine(meth)acrylamides or (meth)acrylates n) are for example epsilon-poly-L-lysine methacrylamide:

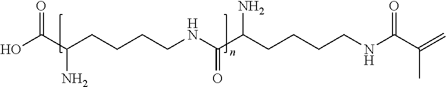

Suitable N-alkyl-4-vinylpridinium and alkyl-2-vinyl-pyridinium salts o) are for example the bromides and iodides of methyl in particular bromides and iodides N-methyl-4-vinylpridinium and N-methyl-2-vinyl-pyridinium.

Suitable biocidal monomers bearing guanide and biguanide groups p) are for example (Meth)acryloyl-modified Poly(hexamethylene biguanide)

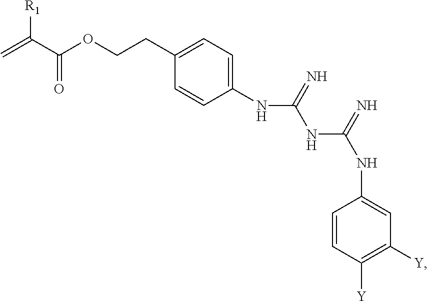

wherein R₁═H, methyl; Y═H, methyl.

Examples of suitable biocidal monomers bearing guanide and biguanide groups include:

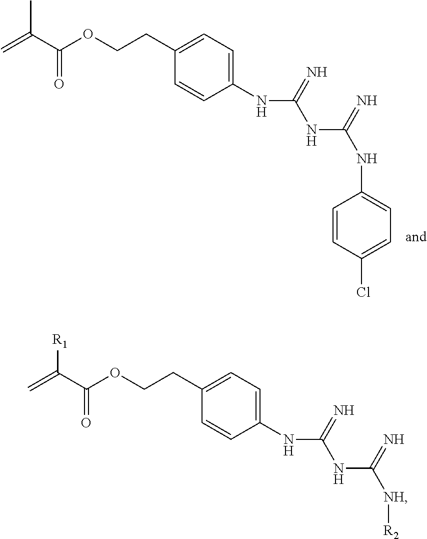

wherein
R₁═H, methyl
R₂=alkyl, aryl, aralkyl, preferentially R₂₌₂-ethyl-hexyl, hexyl, octyl, decyl, dodecyl, tetradecyl, hexadecyl, octadecyl

Suitable halamines q) are for example chloramine

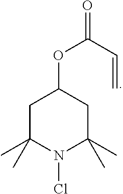 [SMILES: C=CC(=O)OC1CC(C)(C)N(Cl)C(C)(C)C1]

In one embodiment of the invention, polymers according to the invention comprise only antiadhesive monomers as flux enhancing monomers.

In one embodiment of the invention, polymers according to the invention comprise only biocidal monomers as flux enhancing monomers.

In one embodiment of the invention, polymers according to the invention comprise only one antiadhesive monomer and no biocidal monomers.

In one embodiment of the invention, polymers according to the invention comprise only one biocidal monomer and no antiadhesive monomers.

In one embodiment of the invention, polymers according to the invention comprise at least one antiadhesive monomer and at least one biocidal monomer.

Polymers according to the invention may also comprise further monomers having no biocidal or antiadhesive effect.

Suitable further monomers are monomers comprising an ethylenically unsaturated double bond that by themselves do not qualify as flux enhancing monomers a) to q) as defined above. Examples of further monomers include acrylic acid, methacrylic acid, alkyl(meth)acrylate and alkyl(meth)acrylamide, in particular methyl(meth)acrylate, ethyl(meth)acrylate, butyl(meth)acrylate, lauryl(meth)acrylate, ethylhexyl(meth)acrylate, 4-hydroxy butyl(meth)acrylate, phenoxyethyl(meth)acrylate, styrene, alkyl vinyl ether, in particular, methyl vinyl ether, ethyl vinyl ether, n-butyl vinyl ether, 4-hydroxybutyl vinyl ether, vinyl acetate, acrylic nitrile, maleic anhydride.

In one embodiment of the invention, polymers according to the invention comprise at least one antiadhesive and/or biocidal monomer, with the proviso that said at least one antiadhesive and/or biocidal monomer is different from antiadhesive monomers a) as defined above.

In one embodiment of the invention, polymers according to the invention comprise at least one antiadhesive and/or biocidal monomer, with the proviso that said at least one antiadhesive and/or biocidal monomer is not an acrylic ester.

In one embodiment of the invention, polymers according to the invention comprise at least one antiadhesive monomer a) as defined above.

In one embodiment of the invention, polymers according to the invention comprise at least one antiadhesive monomer b)-i) as defined above.

In one embodiment of the invention, polymers according to the invention comprise at least one antiadhesive monomer a) as defined above in combination with at least one antiadhesive and/or biocidal monomer selected from monomers b) to q) as defined above.

In one embodiment of the invention, polymers according to the invention comprise at least one antiadhesive monomer b)-i) as defined above in combination with at least one antiadhesive and/or biocidal monomer selected from monomers c) to q) as defined above.

In one embodiment, polymers according to the invention comprise tBAEMA in combination with at least one flux enhancing monomer comprising at least one quaternary ammonium group.

In another embodiment, polymers according to the invention comprise tBAEMA in combination with at least one halamine.

In another embodiment, polymers according to the invention comprise at least one flux enhancing monomer comprising at least one quaternary ammonium group in combination with at least one halamine.

In one embodiment, polymers according to the invention comprise tBAEMA in combination with at least one flux enhancing monomer comprising at least one quaternary ammonium group and with at least one halamine.

In one embodiment, polymers according to the invention comprise HEMA (2-Hydroxyethyl methacrylate) and QAEMA ([2-(methacryloyloxy)ethyl]trimethylammonium chloride).

In another embodiment polymers according to the invention comprise HEMA (2-Hydroxyethyl methacrylate), QAEMA ([2-(methacryloyloxy)ethyl]trimethylammonium chloride) and acrylic acid.

In a preferred embodiment, polymers according to the invention comprise vinyl pyrrolidone in combination with at least one biocidal monomer j), k), I), m), n), o), p) or q).

In one especially preferred embodiment, polymers according to the invention comprise 2-Isopropenyl-2-oxazoline and at least one monomer according to formula (I)

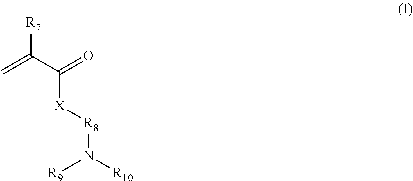

wherein

### R₇ is H or CH₃,

R₉ is C₁-C₅ alkyl bi-radical,
R₉ and R₁₀ are independently H or C₁-C₅ alkyl radical which can be linear or branched,
and X is a divalent radical of —O—, —NH— or —NR₁₁, wherein R₁₁ is C₁-C₅alkyl.

In one particularly preferred embodiment, polymers according to the invention comprise 2-Isopropenyl-2-oxazoline and tBAEMA. In another particularly preferred embodiment, polymers according to the invention comprise 2-Isopropenyl-2-oxazoline and tBAEMA and no further biocidal or antiadhesive monomers as defined above.

In one particularly preferred embodiment, polymers according to the invention comprise 2-isopropenyl-2-oxazoline and vinylpyrrolidone. In another particularly preferred embodiment, polymers according to the invention comprise 2-Isopropenyl-2-oxazoline and vinylpyrrolidone and no further biocidal or antiadhesive monomers as defined above.

Typically, polymers according to the invention comprise 5 to 95% by weight of flux enhancing monomers and 95 to 2 or 95 to 5% by weight of monomers i) and iv) combined (relative to the overall mass of the polymer). In one embodiment polymers according to the invention comprise 50 to 90% by weight, preferably 75% to 90% or 80% to 90% by weight of flux enhancing monomers. In another embodiment, polymers according to the invention comprise 10 to 50% by weight, preferably 20 to 30% by weight of flux enhancing monomers (relative to the overall mass of the polymer).

Polymers according to the invention can be prepared through standard polymerization techniques known to a person skilled in the art.

Polymers according to the invention are normally prepared in a radical polymerization process.

Such radical polymerization process may use radical initiators. Such radical initiators are per se also known in the art.

Preferred radical initiators are azo and peroxo-type initiators, in particular azo initiators.

In another embodiment of the invention, polymers according to the invention are induced in a radiation induced radical polymerization, for example using UV light.

Polymers according to the invention can be prepared in solution or without a solvent. Preferred solvents for the polymerization are water and alcohols in particular water and isopropanol.

Polymers according to the invention can be coated, grafted or otherwise chemically bound to surfaces bearing anchor groups such as carboxylic acid groups that are capable of reacting with the oxazoline or the ring opening products of oxazoline. In another embodiment Polymers according to the invention can be coated and fixed to a surface via physical interactions such as hydrophobic interactions and/or hydrogen bonding.

Polymers according to the invention can thus be coated or grafted onto surfaces like of organic polymers, thus imparting a biocidal and/or antiadhesive effect to that surface.

Polymers according to the invention are useful for applications in the membrane technology. Polymers according to the invention are particularly useful for applications, membranes and apparatuses used for the treatment of water, particularly for the treatment of seawater or brackish water, for the desalination of sea water or brackish water, for the treatment of industrial or municipal wastewater in food processing, or medical applications like dialysis.

Another aspect of the invention is the use of polymers according to the invention or of polymers comprising at least one oxazoline according to formula

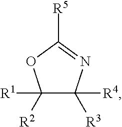

wherein R¹, R², R³ and R⁴ independently denote a hydrogen atom, a halogen atom, an alkyl group, an aralkyl group, a phenyl group, or a substituted phenyl group, and R⁵ denotes a noncyclic organic group having an unsaturated bond reactive in radical polymerization, for enhancing the flux or reducing the decrease of flux over time through membranes.

In another embodiment, such polymers are used for imparting biocidal and/or antiadhesive properties to a membrane.

In another aspect, this invention is directed to membranes, comprising a polymer comprising at least one oxazoline according to formula (O)

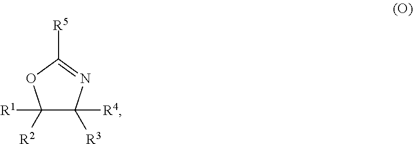

wherein R¹, R², R³ and R⁴ independently denote a hydrogen atom, a halogen atom, an alkyl group, an aralkyl group, a phenyl group, or a substituted phenyl group, and R⁵ denotes a noncyclic organic group having an unsaturated bond reactive in radical polymerization.

In this application the term “membrane” shall, depending on the context, refer to a membrane according to the invention that comprises a polymer comprising at least one oxazoline, or to a membrane that is subjected to a coating with such a polymer to obtain a membrane according to the invention, or both.

Optionally, a membrane or the layer of a membrane that is used as starting material for a coating process to obtain a membrane according to the invention is sometimes referred to as a “base membrane”.

Thus, in case a membrane comprises more than one layer, the “base membrane” can refer to all layers of said membrane as a whole or to each of the layers of said membrane. The term “base membrane” usually refers to the layer that is subjected to the coating with an oxazoline containing polymer.

In one preferred embodiment, the base membrane refers to the separation layer of a membrane.

In another embodiment, the base membrane denotes the support membrane of a membrane, the protective layer or a nonwoven or woven support layer of a membrane.

In a preferred embodiment, suitable membranes and/or the separation layer of a membrane comprise organic polymers, hereinafter referred to as polymers as the main components. A polymer shall be considered the main component of a membrane if it is comprised in said membrane or in the separation layer of said membrane in an amount of at least 50% by weight, preferably at least 60%, more preferably at least 70%, even more preferably at least 80% and particularly preferably at least 90% by weight.

Examples of suitable polymers are polyarylene ether, polysulfone, polyethersulfones (PES), polyphenylenesulfone (PPSU), polyamides (PA), polyvinylalcohol (PVA), cellulose acetate (CA), cellulose diacetate, cellulose triacetate (CTA), CA-triacetate blend, cellulose ester, cellulose nitrate, regenerated cellulose, aromatic, aromatic/aliphatic or aliphatic polyamide, aromatic, aromatic/aliphatic or aliphatic polyimide, polybenzimidazole (PBI), polybenzimidazolone (PBIL), polyacrylonitrile (PAN), polyetheretherketone (PEEK), sulfonated polyetheretherketone (SPEEK), PAN-poly(vinyl chloride) copolymer (PAN-PVC), PAN-methallyl sulfonate copolymer, poly(dimethylphenylene oxide) (PPO), polycarbonate, polyester, polytetrafluroethylene PTFE, poly(vinylidene fluoride) (PVDF), polypropylene (PP), polyelectrolyte complexes, poly(methyl methacrylate) PMMA, polydimethylsiloxane (PDMS), aromatic, aromatic/aliphatic or aliphatic polyimide urethanes, aromatic, aromatic/aliphatic or aliphatic polyamidimides, crosslinked polyimides or mixtures thereof.

Preferably, membranes according to the invention comprise polysulfones, polyethersulfones (PES), polyamides (PA), polyvinylalcohols (PVA), Cellulose Acetate (CA), Cellulose Triacetate (CTA) Poly(vinylidene fluoride) (PVDF) or mixtures thereof as main components.

Suitable polyethersulfones can for example be obtained from BASF SE under the brand name Ultrason®.

Preferred polyarylene ether sulfones (A) are composed of units of the general formula I

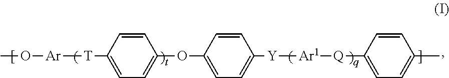

where the definitions of the symbols t, q, Q, T, Y, Ar and Ar¹ are as follows:
t, q: independently of one another 0, 1, 2, or 3,
Q, T, Y: independently of one another in each case a chemical bond or group selected from —O—, —S—, —SO₂—, S═O, C═O, —N═N—, and —CR^{a}R^{b}—, where R^{a} and R^{b} independently of one another are in each case a hydrogen atom or a C₁-C₁₂-alkyl, C₁-C₁₂-alkoxy, or C₆-C₁₈-aryl group, and where at least one of Q, T, and Y is —SO₂—, and
Ar and Ar¹: independently of one another an arylene group having from 6 to 18 carbon atoms.

If, within the abovementioned preconditions, Q, T or Y is a chemical bond, this then means that the adjacent group on the left-hand side and the adjacent group on the right-hand side are present with direct linkage to one another via a chemical bond.

However, it is preferable that Q, T, and Y in formula I are selected independently of one another from —O— and —SO₂—, with the proviso that at least one of the group consisting of Q, T, and Y is —SO₂—.

If Q, T, or Y is —CR^{a}R^{b}—, R^{a} and R^{b} independently of one another are in each case a hydrogen atom or a C₁-C₁₂-alkyl, C₁-C₁₂-alkoxy, or C₆-C₁₈-aryl group.

Preferred C₁-C₁₂-alkyl groups comprise linear and branched, saturated alkyl groups having from 1 to 12 carbon atoms. The following moieties may be mentioned in particular: C₁-C₆-alkyl moiety, e.g. methyl, ethyl, n-propyl, isopropyl, n-butyl, sec-butyl, 2- or 3-methylpentyl, and longer chain moieties, e.g. unbranched heptyl, octyl, nonyl, decyl, undecyl, lauryl, and the singly branched or multibranched analogs thereof.

Alkyl moieties that can be used in the abovementioned C₁-C₁₂-alkoxy groups that can be used are the alkyl groups defined at an earlier stage above having from 1 to 12 carbon atoms. Cycloalkyl moieties that can be used with preference in particular comprise C₃-C₁₂-cycloalkyl moieties, e.g. cyclopropyl, cyclobutyl, cyclopentyl, cyclohexyl, cycloheptyl, cyclooctyl, cyclopropylmethyl, cyclopropylethyl, cyclopropylpropyl, cyclobutylmethyl, cyclobutylethyl, cyclopentylethyl, -propyl, -butyl, -pentyl, -hexyl, cyclohexylmethyl, -dimethyl, and -trimethyl.

Ar and Ar¹ are independently of one another a C₆-C₁₈-arylene group. On the basis of the starting materials described at a later stage below, it is preferable that Ar derives from an electron-rich aromatic substance that is very susceptible to electrophilic attack, preferably selected from the group consisting of hydroquinone, resorcinol, dihydroxynaphthalene, in particular 2,7-dihydroxynaphthalene, and 4,4′-bisphenol. Ar¹ is preferably an unsubstituted C₆- or C₁₂-arylene group.

Particular C₆-C₁₈-arylene groups Ar and Ar¹ that can be used are phenylene groups, e.g. 1,2-, 1,3-, and 1,4-phenylene, naphthylene groups, e.g. 1,6-, 1,7-, 2,6-, and 2,7-naphthylene, and also the arylene groups that derive from anthracene, from phenanthrene, and from naphthacene.

In the preferred embodiment according to formula I, it is preferable that Ar and Ar¹ are selected independently of one another from the group consisting of 1,4-phenylene, 1,3-phenylene, naphthylene, in particular 2,7-dihydroxynaphthylene, and 4,4′-bisphenylene.

Preferred polyarylene ether sulfones (A) are those which comprise at least one of the following repeat units Ia to Io:

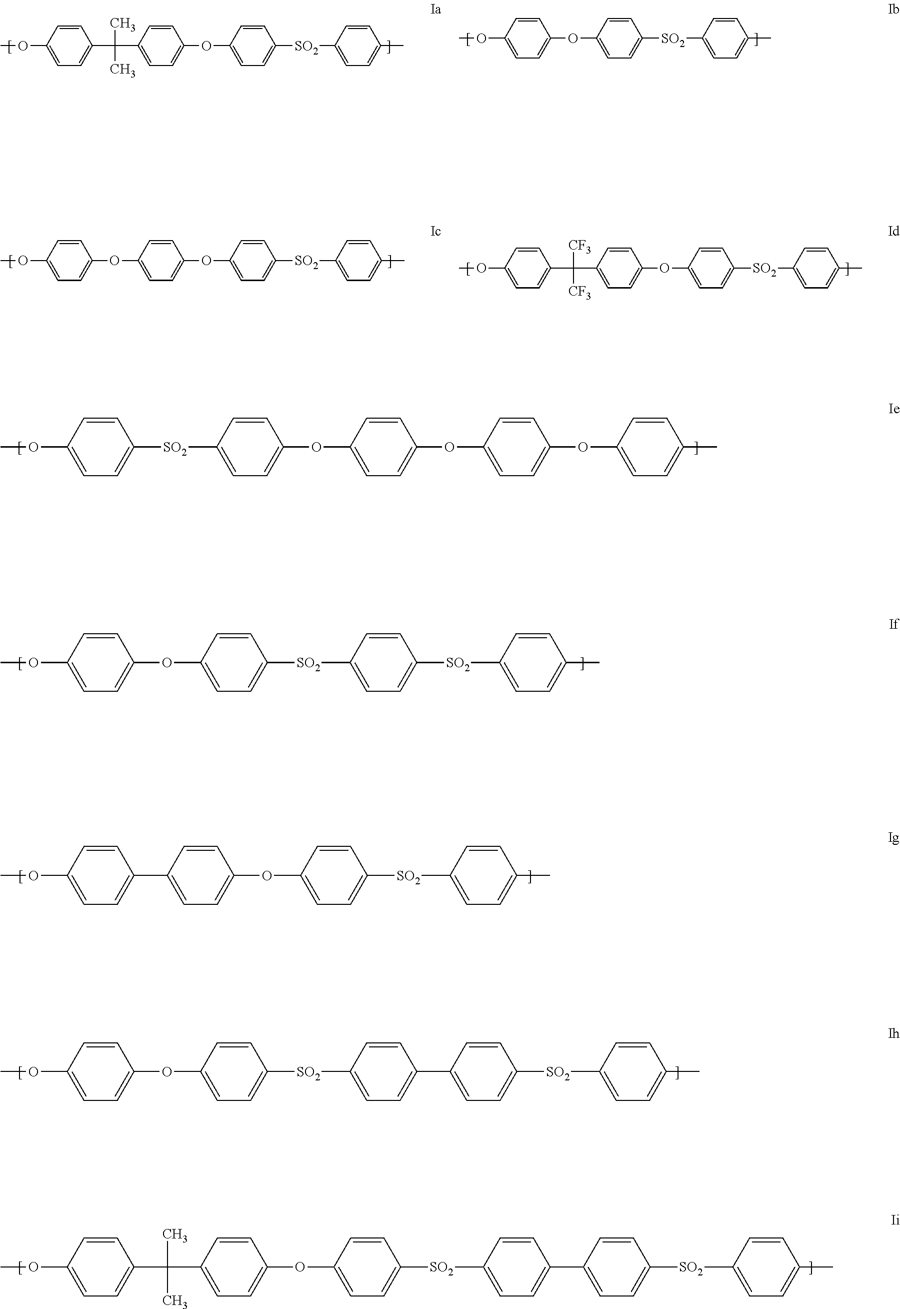

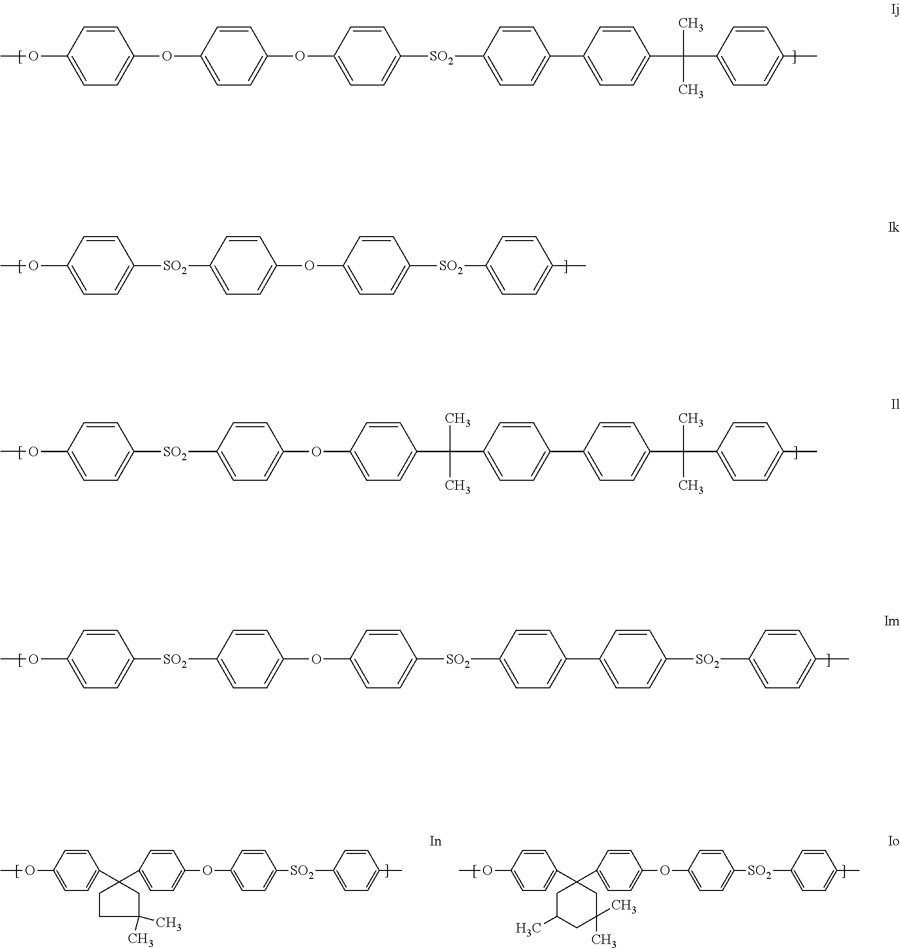

Other preferred units, in addition to the units Ia to Io that are preferably present, are those in which one or more 1,4-phenylene units deriving from hydroquinone have been replaced by 1,3-phenylene units deriving from resorcinol, or by naphthylene units deriving from dihydroxynaphthalene.

Particularly preferred units of the general formula I are the units Ia, Ig, and Ik. It is also particularly preferable that the polyarylene ether sulfones of component (A) are in essence composed of one type of unit of the general formula I, in particular of one unit selected from Ia, Ig, and Ik.

In one particularly preferred embodiment, Ar=1,4-phenylene, t=1, q=0, T is a chemical bond, and Y═SO₂. Particularly preferred polyarylene ether sulfones (A) composed of the abovementioned repeat unit are termed polyphenylene sulfone (PPSU) (formula Ig).

In another particularly preferred embodiment, Ar=1,4-phenylene, t=1, q=0, T=C(CH₃)₂, and Y═SO₂. Particularly preferred polyarylene ether sulfones (A) composed of the abovementioned repeat unit are termed polysulfone (PSU) (formula Ia).

In another particularly preferred embodiment, Ar=1,4-phenylene, t=1, q=0, T=Y═SO₂. Particularly preferred polyarylene ether sulfones (A) composed of the abovementioned repeat unit are termed polyether sulfone (PESU or PES) (formula Ik). This embodiment is very particularly preferred.

For the purposes of the present invention, abbreviations such as PPSU, PESU, and PSU are in accordance with DIN EN ISO 1043-1:2001.

The weight-average molar masses M_{w} of the polyarylene ether sulfones (A) of the present invention are preferably from 10 000 to 150 000 g/mol, in particular from 15 000 to 120 000 g/mol, particularly preferably from 18 000 to 100 000 g/mol, determined by means of gel permeation chromatography in dimethylacetamide as solvent against narrowly-distributed polymethyl methacrylate as standard.

In one embodiment of the invention, suitable polyarylene ether sulfones, particularly polysulfones or polyethersulfones comprise sulfonic acids, carboxylic acid, amino and/or hydroxy groups on some or all of the aromatic rings in the polymer.

Production processes that lead to the abovementioned polyarylene ethers are known to the person skilled in the art and are described by way of example in Herman F. Mark, “Encyclopedia of Polymer Science and Technology”, third edition, volume 4, 2003, chapter “Polysulfones” pages 2 to 8, and also in Hans R. Kricheldorf, “Aromatic Polyethers” in: Handbook of Polymer Synthesis, second edition, 2005, pages 427 to 443.

Suitable membranes are for example membranes suitable as reverse osmosis (RO) membranes, forward osmosis (FO) membranes, nanofiltration (NF) membranes, ultrafiltration (UF) membranes or microfiltration (MF) membranes. These membrane types are generally known in the art.

Suitable membranes are for example those disclosed in US 2011/0027599 in [0021] to [0169]; US 2008/0237126 in col 4, In 36 to col 6, In 3; US 2010/0224555 in [0147] to [0490]; US 2010/0062156 in [0058] to [0225]; US 2011/0005997 in [0045] to [0390], US 2009/0272692 in [0019] to [0073], US 2012/0285890 in [0016] to [0043]; these documents are incorporated herein by reference.

Further suitable membranes are for example those disclosed in U.S. Pat. No. 6,787,216, col. 2, In 54 to col 6, In 19; U.S. Pat. No. 6,454,943, col. 3; In 25 to col. 6, In 12; and WO 2006/012920, p. 3, last paragraph to p. 10, first paragraph.

FO membranes are normally suitable for treatment of seawater, brackish water, sewage or sludge streams. Thereby pure water is removed from those streams through a FO membrane into a so called draw solution on the back side of the membrane having a high osmotic pressure. Typically, FO type membranes, similar as RO membranes are separating liquid mixtures via a solution diffusion mechanism, where only water can pass the membrane whereas monovalent ions and larger components are rejected.

In a preferred embodiment, suitable FO membranes are thin film composite (TFC) FO membranes. Preparation methods and use of thin film composite membranes are principally known and, for example described by R. J. Petersen in Journal of Membrane Science 83 (1993) 81-150.

In a further preferred embodiment, suitable FO membranes comprise a support layer, a separation layer and optionally a protective layer. Said protective layer can be considered an additional coating to smoothen and/or hydrophilize the surface.

Said fabric layer can for example have a thickness of 10 to 500 μm. Said fabric layer can for example be a woven or nonwoven, for example a polyester nonwoven.

Said support layer of a TFC FO membrane normally comprises pores with an average pore diameter of for example 0.5 to 100 nm, preferably 1 to 40 nm, more preferably 5 to 20 nm. Said support layer can for example have a thickness of 5 to 1000 μm, preferably 10 to 200 μm. Said support layer may for example comprise a main component a polysulfone, polyethersulfone, PVDF, polyimide, polyimideurethane or cellulose acetate. Nano particles such as zeolites, particularly zeolite LTA, may be comprised in said support membrane. This can for example be achieved by including such nano particles in the dope solution for the preparation of said support layer.

Said separation layer can for example have a thickness of 0.05 to 1 μm, preferably 0.1 to 0.5 μm, more preferably 0.15 to 0.3 μm. Preferably, said separation layer can for example comprise polyamide or cellulose acetate as the main component.

Optionally, TFC FO membranes can comprise a protective layer with a thickness of 30-500 nm, preferably 100-300 nm. Said protective layer can for example comprise polyvinylalcohol (PVA) as the main component. In one embodiment, the protective layer comprises a halamine like chloramine.

In one preferred embodiment, suitable membranes are TFC FO membranes comprising a support layer comprising polyethersulfone as main component, a separation layer comprising polyamide as main component and optionally a protective layer comprising polyvinylalcohol as the main component.

In a preferred embodiment suitable FO membranes comprise a separation layer obtained from the condensation of a polyamine and a polyfunctional acyl halide. Said separation layer can for example be obtained in an interfacial polymerization process.

RO membranes are normally suitable for removing molecules and ions, in particular monovalent ions. Typically, RO membranes are separating mixtures based on a solution/diffusion mechanism.

In a preferred embodiment, suitable membranes are thin film composite (TFC) RO membranes. Preparation methods and use of thin film composite membranes are principally known and, for example described by R. J. Petersen in Journal of Membrane Science 83 (1993) 81-150.

In a further preferred embodiment, suitable RO membranes comprise a fabric layer, a support layer, a separation layer and optionally a protective layer. Said protective layer can be considered an additional coating to smoothen and/or hydrophilize the surface

Said fabric layer can for example have a thickness of 10 to 500 μm. Said fabric layer can for example be a woven or nonwoven, for example a polyester nonwoven.

Said support layer of a TFC RO membrane normally comprises pores with an average pore diameter of for example 0.5 to 100 nm, preferably 1 to 40 nm, more preferably 5 to 20 nm. Said support layer can for example have a thickness of 5 to 1000 μm, preferably 10 to 200 μm. Said support layer may for example comprise a main component a polysulfone, polyethersulfone, PVDF, polyimide, polyimideurethane or cellulose acetate. Nano particles such as zeolites, particularly zeolite LTA, may be comprised in said support membrane. This can for example be achieved by including such nano particles in the dope solution for the preparation of said support layer.

Said separation layer can for example have a thickness of 0.02 to 1 μm, preferably 0.03 to 0.5 μm, more preferably 0.05 to 0.3 μm. Preferably, said separation layer can for example comprise polyamide or cellulose acetate as the main component.

Optionally, TFC RO membranes can comprise a protective layer with a thickness of 5 to 500 preferable 10 to 300 nm. Said protective layer can for example comprise polyvinylalcohol (PVA) as the main component. In one embodiment, the protective layer comprises a halamine like chloramine.

In one preferred embodiment, suitable membranes are TFC RO membranes comprising a nonwoven polyester fabric, a support layer comprising polyethersulfone as main component, a separation layer comprising polyamide as main component and optionally a protective layer comprising polyvinylalcohol as the main component.

In a preferred embodiment suitable RO membranes comprise a separation layer obtained from the condensation of a polyamine and a polyfunctional acyl halide. Said separation layer can for example be obtained in an interfacial polymerization process.

Suitable polyamine monomers can have primary or secondary amino groups and can be aromatic (e.g. a diaminobenzene, a triaminobenzene, m-phenylenediamine, p-phenylenediamine, 1,3,5-triaminobenzene, 1,3,4-triaminobenzene, 3,5-diaminobenzoic acid, 2,4-diaminotoluene, 2,4-diaminoanisole, and xylylenediamine) or aliphatic (e.g. ethylenediamine, propylenediamine, piperazine, and tris(2-diaminoethyl)amine).

Suitable polyfunctional acyl halides include trimesoyl chloride (TMC), trimellitic acid chloride, isophthaloyl chloride, terephthaloyl chloride and similar compounds or blends of suitable acyl halides. As a further example, the second monomer can be a phthaloyl halide.

In one embodiment of the invention, a separation layer of polyamide is made from the reaction of an aqueous solution of meta-phenylene diamine (MPD)9 with a solution of trimesoyl chloride (TMC) in an apolar solvent.

In another embodiment of the invention, the separation layer and optionally other layers of the membrane contain nanoparticles other than of vanadium pentoxide. Suitable nanoparticles normally have an average particle size of 1 to 1000 nm, preferably 2 to 100 nm, determined by dynamic light scattering. Suitable nanoparticles can for example be zeolites, silica, silicates or aluminium oxide. Examples of suitable nanoparticles include Aluminite, Alunite, Ammonia Alum, Altauxite, Apjohnite, Basaluminite, Batavite, Bauxite, Beideilite, Boehmite, Cadwaladerite, Cardenite, Chalcoalumite, Chiolite, Chloraluminite, Cryolite, Dawsonite, Diaspore, Dickite, Gearksutite, Gibbsite, Hailoysite, Hydrobasaluminite, Hydrocalumite, Hydrotalcite, Illite, Kalinite, Kaolinite, Mellite, Montmoriilonite, Natroalunite, Nontronite, Pachnolite, Prehnite, Prosopite, Ralstonite, Ransomite, Saponite, Thomsenolite, Weberite, Woodhouseite, and Zincaluminit, kehoeite, pahasapaite and tiptopite; and the silicates: hsianghualite, lovdarite, viseite, partheite, prehnite, roggianite, apophyllite, gyrolite, maricopaite, okenite, tacharanite and tobermorite.

Nanoparticles may also include a metallic species such as gold, silver, copper, zinc, titanium, iron, aluminum, zirconium, indium, tin, magnesium, or calcium or an alloy thereof or an oxide thereof or a mixture thereof. They can also be a nonmetallic species such as Si3N4, SiC, BN, B4C, or TIC or an alloy thereof or a mixture thereof. They can be a carbon-based species such as graphite, carbon glass, a carbon cluster of at least C˜, buckminsterfullerene, a higher fullerene, a carbon nanotube, a carbon nanoparticle, or a mixture thereof.

In yet another embodiment the separation layer and optionally other layers of the membrane contain zeolites, zeolite precursors, amorphous aluminosilicates or metal organic frame works (MOFs) any preferred MOFs. Preferred zeolites include zeolite LTA, RHO, PAU, and KFI. LTA is especially preferred.

Preferably, the nanoparticles other than vanadium pentoxide comprised in the membrane have a polydispersity of less than 3.

In another embodiment of the invention the separation layer of the membrane contains a further additive increasing the permeability of the RO membrane. Said further additive can for example be a metal salt of a beta-diketonate compound, in particular an acetoacetonate and/or an at least partially fluorinated beta-diketonate compound.

NF membranes are normally especially suitable for removing separate multivalent ions and large monovalent ions. Typically, NF membranes function through a solution/diffusion or/and filtration-based mechanism.

NF membranes are normally used in cross filtration processes.

NF membranes can for example comprise as the main component polyarylene ether, polysulfone, polyethersulfones (PES), polyphenylensulfone, polyamides (PA), polyvinylalcohol (PVA), Cellulose Acetate (CA), Cellulose Triacetate (CTA), CA-triacetate blend, Cellulose ester, Cellulose Nitrate, regenerated Cellulose, aromatic, aromatic/aliphatic or aliphatic Polyamide, aromatic, aromatic/aliphatic or aliphatic Polyimide, Polybenzimidazole (PBI), Polybenzimidazolone (PBI L), polyetheretherketone (PEEK), sulfonated polyetheretherketone (SPEEK), Polyacrylonitrite (PAN), PAN-poly(vinyl chloride) copolymer (PAN-PVC), PAN-methallyl sulfonate copolymer, Polysulfone, Poly(dimethylphenylene oxide) (PPO), Polycarbonate, Polyester, Polytetrafluroethylene PTFE, Poly(vinylidene fluoride) (PVDF), Polypropylene (PP), Polyelectrolyte complexes, Poly(methyl methacrylate) PMMA, Polydimethylsiloxane (PDMS), aromatic, aromatic/aliphatic or aliphatic polyimide urethanes, aromatic, aromatic/aliphatic or aliphatic polyamidimides, crosslinked polyimides or mixtures thereof. In a preferred embodiment, said main components of NF membranes are positively or negatively charged.

Nanofiltration membranes often comprise charged polymers comprising sulfonic acid groups, carboxylic acid groups and/or ammonium groups.

Preferably, NF membranes comprise as the main component polyamides, polyimides or polyimide urethanes, Polyetheretherketone (PEEK) or sulfonated polyetheretherketone (SPEEK).

UF membranes are normally suitable for removing suspended solid particles and solutes of high molecular weight, for example above 1000 Da. In particular, UF membranes are normally suitable for removing bacteria and viruses.

UF membranes normally have an average pore diameter of 0.5 nm to 50 nm, preferably 1 to 40 nm, more preferably 5 to 20 nm.

UF membranes can for example comprise as main component a polyarylene ether, polysulfone, polyethersulfones (PES), polyphenylensulfone (PPSU), polyamides (PA), polyvinylalcohol (PVA), Cellulose Acetate (CA), Cellulose Triacetate (CTA), CA-triacetate blend, Cellulose ester, Cellulose Nitrate, regenerated Cellulose, aromatic, aromatic/aliphatic or aliphatic Polyamide, aromatic, aromatic/aliphatic or aliphatic Polyimide, Polybenzimidazole (PBI), Polybenzimidazolone (PBIL), Polyacrylonitrile (PAN), PAN-poly(vinyl chloride) copolymer (PAN-PVC), PAN-methallyl sulfonate copolymer, Polysulfone, Poly(dimethylphenylene oxide) (PPO), Polycarbonate, Polyester, Polytetrafluroethylene PTFE, Poly(vinylidene fluoride) (PVDF), Polypropylene (PP), Polyelectrolyte complexes, Poly(methyl methacrylate) PMMA, Polydimethylsiloxane (PDMS), aromatic, aromatic/aliphatic or aliphatic polyimide urethanes, aromatic, aromatic/aliphatic or aliphatic polyamidimides, crosslinked polyimides or mixtures thereof.

Preferably, UF membranes comprise as main component polysulfone, polyethersulfone, polyphenylenesulfone, PVDF, polyimide, polyamidimide, crosslinked polyimides, polyimide urethanes or mixtures thereof.

In one embodiment, UF membranes comprise further additives like polyvinyl pyrrolidones.

In one embodiment, UF membranes comprise further additives like block copolymers of polyarylene sulfones and alkyleneoxides like polyethyleneoxide.

In a preferred embodiment, UF membranes comprise as major components polysulfones or polyethersulfone in combination with further additives like polyvinylpyrrolidone.

In one preferred embodiment, UF membranes comprise 80 to 50% by weight of polyethersulfone and 20 to 50% by weight of polyvinylpyrrolidone.

In another embodiment UF membranes comprise 95 to 80% by weight of polyethersulfone and 5 to 15% by weight of polyvinylpyrrolidone.

In another embodiment UF membranes comprise 99.9 to 80% by weight of polyethersulfone and 0.1 to 15% by weight of polyvinylpyrrolidone.

In one embodiment of the invention, UF membranes are present as spiral wound membranes.

In another embodiment of the invention, UF membranes are present as tubular membranes.

In another embodiment of the invention, UF membranes are present as flat sheet membranes.

In another embodiment of the invention, UF membranes are present as hollow fiber membranes.

In yet another embodiment of the invention, UF membranes are present as single bore hollow fiber membranes.

In yet another embodiment of the invention, UF membranes are present as multi bore hollow fiber membranes.

MF membranes are normally suitable for removing particles with a particle size of 0.1 μm and above.

MF membranes normally have an average pore diameter of 0.1 μm to 10 μm, preferably 1.0 μm to 5 μm.

Microfiltration can use a pressurized system but it does not need to include pressure.

MF membranes can be hollow fibers, flat sheet, tubular, spiral wound, hollow fine fiber or track etched. They are porous and allow water, monovalent species (Na+, Cr), dissolved organic matter, small colloids and viruses through while retaining particles, sediment, algae or large bacteria.

Microfiltration systems are designed to remove suspended solids down to 0.1 micrometres in size, in a feed solution with up to 2-3% in concentration.

MF membranes can for example comprise as main component polyarylene ether, polysulfone, polyethersulfones (PES), polyphenylensulfone (PPSU), polyamides (PA), polyvinylalcohol (PVA), Cellulose Acetate (CA), Cellulose Triacetate (CTA), CA-triacetate blend, Cellulose ester, Cellulose Nitrate, regenerated Cellulose, aromatic, aromatic/aliphatic or aliphatic Polyamide, aromatic, aromatic/aliphatic or aliphatic Polyimide, Polybenzimidazole (PBI), Polybenzimidazolone (PBIL), Polyacrylonitrile (PAN), PAN-poly(vinyl chloride) copolymer (PAN-PVC), PAN-methallyl sulfonate copolymer, Polysulfone, Poly(dimethylphenylene oxide) (PPO), Polycarbonate, Polyester, Polytetrafluroethylene PTFE, Poly(vinylidene fluoride) (PVDF), Polypropylene (PP), Polyelectrolyte complexes, Poly(methyl methacrylate) PMMA, Polydimethylsiloxane (PDMS), aromatic, aromatic/aliphatic or aliphatic polyimide urethanes, aromatic, aromatic/aliphatic or aliphatic polyamidimides, crosslinked polyimides or mixtures thereof.

Under the conditions applied for coating of grafting the surface of a base membrane with polymers useful according to the invention, the oxazoline rings comprised in the polymer may partially or completely open in a nucleophilic addition, hydrolysis or particularly acidolysis reaction. In this application and in the context of membranes comprising the above polymer comprising an oxazoline, the term “polymer” and “oxazoline” shall refer to said polymer comprising oxazoline in the form as depicted in formula (0), as well as polymers or oxazolines, in which the ring structure has opened and optionally reacted with an acidic group like a carboxylate, sulfonic acid, phosphoric acid or phosphonic acid group or a thiol group present on the surface of the base membrane or in the coating mixture.

Preferably membranes according to the invention comprise a polymer wherein said at least one oxazoline is selected from 2-Isopropenyl-2-oxazolin, 2-vinyl-2-oxazoline, 2-vinyl-4-methyl-2-oxazoline, 2-vinyl-5-methyl-2-oxazoline, 2-isopropenyl-2-oxazoline, 2-isopropenyl-4-methyl-2-oxazoline and 2-isopropenyl-5-ethyl-2-oxazoline.

Most preferably membranes according to the invention comprise a polymer wherein said oxazoline is 2-Isopropenyl-2-oxazolin

In a preferred embodiment, membranes according to the invention comprise a polymer, which comprises
i) at least one oxazoline according to formula

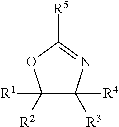

wherein R¹, R², R³ and R⁴ independently denote a hydrogen atom, a halogen atom, an alkyl group, an aralkyl group, a phenyl group, or a substituted phenyl group, and R⁵ denotes a noncyclic organic group having an unsaturated bond reactive in radical polymerization,
ii) optionally at least one antiadhesive monomer,
iii) optionally at least one biocidal monomer,
iv) optionally at least one further monomer,
wherein said polymer comprises at least one monomer selected from monomers ii) and iii),

Membranes according to the invention comprise a polymer that has been coated on the surface of a base membrane. Said polymer can bind to the surface of the base membrane through adhesion or, preferably, through covalent bonds with the surface of the base membrane.

Monomers that impart flux enhancing properties to the membrane are herein also referred to as “flux enhancing monomers”. The term “flux” shall denote the flux of the medium that is subjected to a separation operation. In many cases, “flux” means the flux of water through the membrane. For example in the case of water treatment applications, “flux” means the amount of water that permeates through the specified membrane area in a certain period of time. Flux enhancing properties in the context of this invention refer in particular to the long term properties of membranes. While it is possible that through the application of a coating the flux may decrease over a short term, the flux over the long term will be improved (meaning that the decrease of flux is reduced) relative to a membrane to that no such coating has been applied. The duration of a “short term” or “long term” may vary depending on the membrane or the application or the material subjected to that application, that is for example from the type of water treated. Thus, enhancing of flux in the context of this application shall mean that after at least one certain period of time and under at least one set of application conditions, the flux through a membrane according to the invention shall be improved or the decrease of flux be reduced over the flux through a membrane comprising no coating according to this invention or over membranes known from the art. For example, membranes according to the invention may show improved flux over prior art membranes after a period of 1 hour, 1 day, 3 days, 5 days, 1 week, 2 weeks, three weeks, one month, two months, three months, six months and/or one year. Sometimes the enhanced flux of membranes according to the invention only becomes observable after one or a certain number of cleaning cycles have been applied to the membrane.

In particular, suitable flux enhancing monomers reduce fouling and in particular biofouling of the membrane.

In the context of this application, an effect of a polymer or the coating comprising a flux enhancing monomer is also sometimes referred to as the effect of the flux enhancing monomer.

Monomers bearing a charge, for example from ammonium groups or carboxylate groups, are accompanied by one or more counterions. If, in this application, a monomer bearing a charge is depicted or named without corresponding counterion, such monomers are to be understood to be accompanied by a suitable counterion (with the exception of betaines). Such counterions are for example chloride, bromide, iodide, carboxylates for monomers bearing a positive charge. For monomers bearing negative charge, suitable counterions are for example sodium, potassium, magnesium, calcium, ammonium.

In a preferred embodiment, suitable flux enhancing monomers are antiadhesive or biocidal monomers that impart biocidal and/or antiadhesive properties to the membrane.

In a preferred embodiment, membranes according to the invention comprise a polymer, which comprises
i) at least one oxazoline according to formula

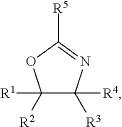

wherein R¹, R², R³ and R⁴ independently denote a hydrogen atom, a halogen atom, an alkyl group, an aralkyl group, a phenyl group, or a substituted phenyl group, and R⁵ denotes a noncyclic organic group having an unsaturated bond reactive in radical polymerization,
ii) optionally at least one antiadhesive monomer,
iii) optionally at least one biocidal monomer,
iv) optionally at least one further monomer,
wherein said polymer comprises at least one monomer selected from monomers ii) and iii).

Suitable antiadhesive and biocidal monomers are those disclosed above.

In one embodiment of the invention, membranes according to the invention comprise at least one antiadhesive monomer selected from
a) esters of (meth)acrylic acid polyols
b) vinyl ethers of polyols
c) hydrophilic macromonomers different from monomers a) and b)
d) N-vinyl compounds
e) Low molecular weight hydrophilic (meth)acrylamides
f) (meth)acrylates or (meth)acrylamides bearing epoxy groups
g) monomers having a betain structure
h) hydrophilic monomers different from those mentioned under a) to g)
i) Ion pair comonomers.

Antiadhesive monomers a) to i) mean those antiadhesive monomers as defined above.

In one embodiment of the invention, membranes according to the invention comprise at least one biocidal monomer is selected from
j) vinyl-imidazolium compounds
k) ethylenically unsaturated monomers bearing quarternary ammonium or phosphonium groups
l) diallyldialkylammoniumchlorides
m) alkylaminoalkyl(meth)acrylates and alkylaminoalkyl(meth)acrylamides
n) Polylysine(meth)acrylamides or (meth)acrylates
o) alkyl-4-vinylpridinium and alkyl-2-vinyl-pyridinium salts, in particular bromides and iodides
p) ethylenically unsaturated monomers bearing guanide and biguanide groups
q) Halamines.

Antiadhesive and biocidal monomers a) to q) mean those antiadhesive and biocidal monomers as defined above accordingly.

In one embodiment of the invention, membranes comprise a coating comprising only one antiadhesive monomer and no biocidal monomer as flux enhancing monomer.

In one embodiment of the invention, membranes comprise a coating comprising only one biocidal monomer and no antiadhesive monomer as flux enhancing monomer.

In one embodiment of the invention, membranes comprise a coating comprising at least one antiadhesive and at least one biocidal monomer as flux enhancing monomers.

In one embodiment of the invention, membranes according to the invention comprise at least one antiadhesive and/or biocidal monomer, with the proviso that said at least one antiadhesive and/or biocidal monomer is different from antiadhesive monomers a) as defined above.

In one embodiment of the invention, membranes according to the invention comprise at least one antiadhesive and/or biocidal monomer, with the proviso that said at least one antiadhesive and/or biocidal monomer is not an acrylic ester.

In one embodiment of the invention, membranes according to the invention comprise at least one antiadhesive monomer a) as defined above.

In one embodiment of the invention, membranes according to the invention comprise at least one antiadhesive monomer b)-i) as defined above.

In one embodiment of the invention, membranes according to the invention comprise at least one antiadhesive monomer a) as defined above in combination with at least one antiadhesive and/or biocidal monomer selected from monomers b) to q) as defined above.

In one embodiment of the invention, membranes according to the invention comprise at least one antiadhesive monomer b)-i) as defined above in combination with at least one antiadhesive and/or biocidal monomer selected from monomers c) to q) as defined above.

In one embodiment of the invention, membranes according to the invention comprise at least one antiadhesive monomer a) as defined above in combination with at least one antiadhesive and/or biocidal monomer selected from monomers b) to q) as defined above.

In one embodiment of the invention, membranes according to the invention comprise at least one antiadhesive monomer b) to i) as defined above.

In one embodiment, membranes according to the invention comprise tBAEMA in combination with at least one flux enhancing monomer comprising at least one quaternary ammonium group. In another embodiment, membranes according to the invention comprise tBAEMA in combination with at least one halamine.

In another embodiment, membranes according to the invention comprise at least one flux enhancing monomer comprising at least one quaternary ammonium group in combination with at least one halamine.

In one embodiment, membranes according to the invention comprise tBAEMA in combination with at least one flux enhancing monomer comprising at least one quaternary ammonium group and with at least one halamine.

In one embodiment, membranes according to the invention comprise a coating comprising HEMA (2-Hydroxyethyl methacrylate) and QAEMA ([2-(methacryloyloxy)ethyl]trimethylammonium chloride).

In another embodiment, membranes according to the invention comprise a coating comprising HEMA (2-Hydroxyethyl methacrylate), QAEMA ([2-(methacryloyloxy)ethyl]trimethylammonium chloride) and acrylic acid.

In another preferred embodiment, membranes according to the invention comprise vinyl pyrrolidone in combination with at least one biocidal monomer j), k), I), m), n), o), p) or q).

Polymers useful according to the invention comprising oxazoline and at least one flux enhancing monomer are typically coated or grafted onto the outermost layer of a base membrane facing the feed side of the membrane to obtain membranes according to the invention. The coated or grafted polymer normally has a thickness of 1 nm to 100 μm, preferably 5 nm to 300 nm, most preferably 10 nm to 100 nm.

Polymers useful according to the invention can be applied on the base membrane neat or in a formulation with a solvent.

In one embodiment of the invention, membranes according to the invention are made in a process comprising
A) coating the surface of a base membrane with a formulation comprising
   I) a polymer useful according to the invention,
   II) optionally at least one di- or polycarboxylic acid, di- or polysulfonic acid, di- or polyphosphonic acid, di- or poly phosphoric acid or components comprising two or more of these acid groups and/or thiol groups or latent acids, di- or polyacids that form the acid during the coating process. Preferred is a poly(meth)acrylic acid or the ammonium salt of poly(meth)acrylic acid as a latent polyacid that upon coating and annealing loses ammonia, thereby forming poly(meth)acrylic acid that can further react with the oxazoline ring;
B) optionally annealing of the coating and
C) optionally extracting nonreacted components from I) and/or II).

In another embodiment of the invention, membranes according to the invention are made in a process comprising coating the surface of a base membrane with two or more formulations, each comprising at least one of components I) to II), wherein at least one formulation comprises component I). For example a base membrane can be treated with one formulation comprising component I) and another formulation comprising components II. If components I) to II) are applied to the base membrane in more than one formulation, this can be done simultaneously or subsequentially and optionally followed by annealing the coating and optionally followed by extracting nonreacted components from I) and/or II).

In another embodiment of the invention, membranes according to the invention are made in a process comprising treating a base membrane with a formulation comprising components I), wherein said formulation does not comprise a component II), optionally followed by annealing the coating and optionally followed by extracting nonreacted components from I). In a second step said base membrane is treated with a formulation comprising component I and optionally II.

Said formulation can optionally comprise at least one di- or polycarboxylic acid, di- or polysulfonic acid, di- or polyphosphonic acid, di- or poly phosphoric acid or components comprising two or more of these acid groups and/or thiol groups. Preferably said di- or polycarboxylic acid is a polyacrylic acid.

In one embodiment of the invention, the coating obtained in the above process is annealed by exposing the coated membrane to elevated temperatures. For example, the coated membrane can be heated to a temperature of 40 to 130° C., for a period of 30 seconds to 5 hours.

In another embodiment of the invention, the coated membrane is not annealed by heating.

Said formulation may comprise one or more solvents. Examples of suitable solvents are water, THF, dioxane, alcohols or mixtures thereof. Preferred solvents are water or alcohols, in particular water or isopropanol or mixtures thereof. A preferred solvent is water. In a preferred embodiment of the present invention said polymer is comprised in the formulation in a concentration in the range of from 0.01 to 70% by weight, more preferably in the range of from 0.5 to 60% by weight, based on the total weight of the formulation.

In one embodiment, the composition or formulation comprising the at least one flux enhancing monomer optionally comprises further additives like dispersants. Further additives that can be comprised generally are known in the art.

Some types of membranes by themselves comprise anchor groups on the surface of the membrane. Examples of such membranes include polyamide membranes like RO or FO membranes with a separation layer based on polyamide.

Anchor groups in this context means a functional group that is capable of reacting with oxazoline, thus binding the polymer to the surface of the base membrane. Suitable anchor groups include for example carboxylic groups, sulfonic acid groups, phosphonic acid, phosphoric acid and thiols.

These types of membranes comprising by themselves anchor groups can bind to the polymer comprising oxazoline in a reaction between said acidic groups on the surface of the membrane and oxazoline groups comprised in the polymer.

Some types of membranes do not by themselves comprise anchor groups on the surface of the membrane. Examples of such membranes include membranes based on polysulfones, polyethersulfones, cellulose acetate or PVDF.

In one embodiment of the invention, particularly if the base membrane does not by itself comprise anchor groups on the surface of the membrane, the surface of said membrane can be subjected to additional process steps to obtain anchor groups on the surface of the base membrane.

In one embodiment of the invention the surface of the base membrane is subjected to an oxidative process like flame treatment, corona discharge, plasma treatment, in particular oxygen-containing plasma, actinic irradiation such as ultraviolet, x-ray or gamma irradiation and electron beam treatment, treatment with oxidative immersion baths such as baths containing chromium sulfuric acid, sulfuric acid, hydrogen peroxide ammonium hydroxide, persulfuric acid, peroxo disulfuric acid, phosphoric acid, hypophosphorous acid, phosphorous acid, pyrophosphoric acid, triphosphoric acid, perphosphoric acid, permonophosphoric acid and mixtures thereof.

Corona discharges can be electrical discharges characterized by a corona and occurring when one of two electrodes in a gas has a shape causing the electric field at its surface to be significantly greater than that between the electrodes. Air is usually used as gas. The substrate is usually located at ambient pressure in the discharge field between the two electrodes, for example by passing a film as substrate between two electrodes.

Plasma can be a gas where electrons and ions are present. Plasma can be generated by the treatment of gases with high temperatures or high electric fields. Plasma treatment is usually carried out in vacuum chambers at 10 to 100 Pa with a nonthermal plasma in a gas atmosphere consisting of an inert gas or reactive gas, for example oxygen.

Flame can be flames that are formed when a flammable gas and an oxygen containing gas, for example atmospheric air, are combined and combusted. Examples of flammable gases are propane, butane or town gas. Flame treatment is usually carried out at ambient pressure.

Ozone can be generated from atmospheric oxygen in a corona discharge or by ultraviolet radiation.

Electron beam can be generated by electron beam accelerators, for example by cathode ray tubes.

X-rays can be generated by X-ray generators, for example by X-ray-tubes.

Preferably, the oxidation of the surface is performed by treatment with corona discharge, plasma or flame. More preferably, it is performed by corona discharge treatment or plasma treatment.

In another embodiment of the invention the surface of the base membrane is subjected to a non-oxidative process like physical deposition of molecules, in particular polymers, containing anchor groups, the formation of interpenetrating networks of the membrane with polymers containing anchor groups or and the formation of self-assembled monolayers of anchor group containing molecules on the membrane surface.

One aspect of the invention is thus a process for making membranes according to the invention comprising at least one of the following steps:
D) surface modification of the base membrane using at least one oxidative process to obtain anchor groups, said oxidative process preferably being flame treatment, oxidative immersion baths such as baths containing chromium sulfuric acid, sulfuric acid, hydrogen peroxide ammonium hydroxide, persulfuric acid, peroxo disulfuric acid, phosphoric acid, hypophosphorous acid, phosphorous acid, pyrophosphoric acid, triphosphoric acid, perphosphoric acid, permonophosphoric acid and mixtures thereof corona discharge, plasma treatment in particular oxygen-containing plasma, actinic irradiation such as ultraviolet, x-ray or gamma irradiation and electron beam treatment,
E) surface modification of the base membrane using a non-oxidative process to obtain anchor groups, said non-oxidative process preferably being physical deposition of molecules, in particular polymers, containing said anchor groups, the formation of interpenetrating networks of the membrane with polymers containing said anchor groups and the formation of self-assembled monolayers of such anchor group containing molecules on the membrane surface.

In some cases anchor groups are not needed since adhesion of the coating by physical interactions such as hydrophobic interactions, pi-pi interactions and/or hydrogen bonding are strong enough.

Another aspect of the invention is a process for making membranes comprising the steps
A) coating the surface of a base membrane with a formulation comprising
   I) a polymer useful according to the invention,
   II) optionally at least one di- or polycarboxylic acid, di- or polysulfonic acid, di- or polyphosphonic acid, di- or poly phosphoric acid or components comprising two or more of these acid groups and/or thiol groups or latent acids, di- or polyacids that form the acid during the coating process. Preferred is a poly(meth)acrylic acid or the ammonium salt of poly(meth)acrylic acid as a latent polyacid that upon coating and annealing loses ammonia, thereby forming poly(meth)acrylic acid that can further react with the oxazoline ring;
B) optionally annealing of the coating and
C) optionally extracting nonreacted components from I) and/or II).

Another aspect of the invention is a method of improving the flux through membranes, which comprises coating the surface of a base membrane in a process comprising the steps
A) coating the surface of a base membrane with a formulation comprising
   I) a polymer useful according to the invention,
   II) optionally at least one di- or polycarboxylic acid, di- or polysulfonic acid, di- or polyphosphonic acid, di- or poly phosphoric acid or components comprising two or more of these acid groups and/or thiol groups or latent acids, di- or polyacids that form the acid during the coating process. Preferred is a poly(meth)acrylic acid or the ammonium salt of poly(meth)acrylic acid as a latent polyacid that upon coating and annealing loses ammonia, thereby forming poly(meth)acrylic acid that can further react with the oxazoline ring;
B) optionally annealing of the coating and
C) optionally extracting nonreacted components from I) and/or II).

Another aspect of the invention is a composition comprising a polymer useful according to the invention, optionally at least one di- or polycarboxylic acid, di- or polysulfonic acid, di- or polyphosphonic acid, di- or poly phosphoric acid or components comprising two or more of these acid groups and/or thiol groups or latent acids, di- or polyacids that form the acid during the coating process and optionally at least one solvent.

Another aspect of the invention is the use of a composition comprising a polymer useful according to the invention, optionally at least one di- or polycarboxylic acid, di- or polysulfonic acid, di- or polyphosphonic acid, di- or poly phosphoric acid or components comprising two or more of these acid groups and/or thiol groups or latent acids, di- or polyacids that form the acid during the coating process and optionally at least one solvent for improving the flux through membranes, or for imparting biocidal and/or antiadhesive properties to a membrane.

Membranes according to the invention show improved properties with respect to the decrease of flux over time and their fouling and particularly biofouling properties.

Membranes according to the invention are easy and economical to make.

Filtration systems and membranes according to the invention can be made using aqueous or alcoholic systems and are thus environmentally friendly. Furthermore, leaching of toxic substances is not problematic with membranes according to the invention.

Membranes according to the invention have a long lifetime and allow for the treatment of water.

Membranes according to the invention can be cleaned more easily and with lower amounts of cleaning agents than membranes known from the art.

Membranes according to the invention have longer cleaning cycles meaning that they need to be cleaned less often than membranes known from the art.

In a preferred embodiment, membranes according to the invention are used for the treatment of sea water or brackish water.

In one preferred embodiment of the invention, membranes according to the invention, particularly RO, FO or NF membranes are used for the desalination of sea water or brackish water.

Membranes according to the invention, particularly RO, FO or NF membranes are used for the desalination of water with a particularly high salt content of for example 3 to 8% by weight. For example membranes according to the invention are suitable for the desalination of water from mining and oil/gas production and fracking processes, to obtain a higher yield in these applications.

Different types of membrane according to the invention can also be used together in hybrid systems combining for example RO and FO membranes, RO and UF membranes, RO and NF membranes, RO and NF and UF membranes, NF and UF membranes.

In another preferred embodiment, membranes according to the invention, particularly NF, UF or MF membranes are used in a water treatment step prior to the desalination of sea water or brackish water.

In another preferred embodiment membranes according to the invention, particularly NF, UF or MF membranes are used for the treatment of industrial or municipal waste water.

Membranes according to the invention, particularly RO and/or FO membranes can be used in food processing, for example for concentrating, desalting or dewatering food liquids (such as fruit juices), for the production of whey protein powders and for the concentration of milk, the UF permeate from making of whey powder, which contains lactose, can be concentrated by RO, wine processing, providing water for car washing, making maple syrup, during electrochemical production of hydrogen to prevent formation of minerals on electrode surface, for supplying water to reef aquaria

Membranes according to the invention, particularly UF membranes can be used in medical applications like, dialysis and other blood treatments, food processing, concentration for making cheese, processing of proteins, desalting and solvent-exchange of proteins, fractionation of proteins, clarification of fruit juice, recovery of vaccines and antibiotics from fermentation broth, laboratory grade water purification, drinking water disinfection (including removal of viruses), removal of endocrines and pesticides combined with suspended activated carbon pretreatment.

Membranes according to the invention, particularly RO, FO, NF membranes can be used for rehabilitation of mines, homogeneous catalyst recovery, desalting reaction processes.

Membranes according to the invention, particularly NF membranes, can be used for separating divalent ions or heavy and/or radioactive metal ions, for example in mining applications, homogeneous catalyst recovery, desalting reaction processes.

### EXAMPLES

### Determination of Antiadhesive Properties (Proteins)

RO membranes were painted black at the macroporous backside. Pieces of 9 mm in diameter were punched out and put into a 48 well plate. Into each well, 500 μL of buffer solution (10 mmol/l HEPES, pH 7.4) was added and the samples equilibrated for 30 min. Then 100 μL of the buffer solution were replaced with 100 μL of a solution of 0.2 g/I fluorescently-labelled fibrinogen (from human plasma, AlexaFluor® 647 Conjugate, Molecular Probes®) in buffer (10 mmol/l HEPES, pH 7.4) and the samples equilibrated for 2 hours at 30° C. Subsequently, the samples were rinsed by 5 times replacing 400 μL of the 500 μL solution in each well with 400 μL pure buffer (10 mmol/l HEPES, pH 7.4). Samples were then transferred to a new 48 well plate and covered with 500 μL of buffer solution (10 mmol/l HEPES, pH 7.4). The well plates were analyzed in a microarray fluorescence scanner.

### Determination of Antiadhesive Properties (Bacteria):

Coated membranes were tested against bacterial adhesion (Styphylococcus aureus). The membrane was cut and sealed in a holder such that only the coated upper surface was accessible to liquids. The coated surface was then covered with approximately 1 ml of a bacterial suspension (Staphylococcus aureus, OD600˜1, in 0.5% TSBY/0.9% NaCl supplemented with Syto9® and propidium iodide fluorescent dyes as specified by the supplier (Film Tracer Live/Dead® Biofilm Viability Kit, Invitrogen)). After incubation of the bacteria on the surface for one hour at 37° C., planktonic cells were rinsed off by repeated (10 times) exchange of 90% of the liquid supernatant with bacteria-free 0.9% NaCl solution. This way, the membrane surface was kept moist during all steps of the procedure. Bacteria attached to the membrane surface (sessile cells) were then detected and enumerated either by fluorescence microscopy or by punching out a small piece, followed by bacteria recovery by ultrasonication and serial dilution plating.

### Determination of Antimicrobial Activity:

Antimicrobial activity of coated membranes was determined either by testing according to ISO 22196 (JIS Z2801) or by a fluorescence microscopy assay as detailed below:

### 1. Bacterial Culture:

50 ml of DSM 92 medium (=TSBY Medium, Deutsche Sammlung von Mikroorganismen and Zellkulturen GmbH) in an Erlenmeyer flask with chicane are inoculated with a single colony of Staphylococcus aureus ATCC 6538P and incubated at 190 rpm and 37° C. for 16 hours. The resulting preliminary culture has a cell density of approximately 108 CFU/ml, corresponding to an optical density of OD=7.0-8.0. Using this preliminary culture, 15 ml of main culture in 5% DSM 92 medium with an optical density of OD=1.0 are prepared.

### 2. Fluorescence Staining:

500 μl of the main bacterial culture are stained in accordance with the manufacturer recommendation using 1.5 μl of Syto 9 fluorescent dye and 1.5 μl of propidium iodide fluorescent dye (Film Tracer™ LIVE/DEAD® Biofilm Viability Kit, from Invitrogen). 10 μl of this bacterial suspension are applied to the surface under investigation, and covered with a cover slip. A homogeneous film of liquid is formed, with a thickness of about 30 μm. The test substrates are incubated in the dark at 37° C. for up to 2 hours. After this time, >95% living bacterial cells are found on untreated reference substrates (including pure glass).

### 3. Microscopy:

The test substrates are examined under a Leica DMI6000 B microscope with the cover slip facing the lens. Each test substrate is advanced automatically to 15 pre-defined positions, and images are recorded in the red (R) and green (G) fluorescence channel. The absorbance and emission wavelengths in the fluorescence channels are adapted to the dyes used. Bacteria with an intact cell membrane (living) are detected in the green channel, bacteria with a defective cell membrane (dead) are detected in the red channel. For each of the 15 positions, the number of bacteria in both channels is counted. The percentage of dead bacteria is calculated from the numbers in R/(R+G). The percentage of dead bacteria is averaged over the 15 positions and reported as the result.

### Example 1

### Preparation of Copolymer X1 (QVI-C16:iPDX 1:4)

319 parts by weight of water and 10 parts by weight of 1-hexadecyl-3-vinyl-imidazol-1-ium bromide were mixed under nitrogen and heated to 60° C. 1.1 parts by weight of 2,2′-Azobis(2-methylpropionamidine)dihydrochloride (Wako V 50, Wako Chemicals GmbH, Germany) were mixed with 63 parts by weight of water and added within one hour at 60° C. During the same time 11.1 parts by weight of 2-isopropenyl-2-oxazoline were added. After addition of all components, the mixture was kept at 60° C. for 9 hours.

An opaque dispersion was obtained. GPC analysis (solvent DMAC, polyester copolymer column; calibration against PMMA standards of PSS Polymer Standards Service GmbH, Germany) revealed a number average molecular weight Mn of 32900 g/mol, a weight average molecular weight Mw of 123300 g/mol and a polydispersity index PDI of 3.7. 2-isopropenyl-2-oxazoline rest monomer content was 10ppm.

### Example 2

### Preparation of Copolymer X2 (QVI-C16:iPDX 1:1)

420 parts by weight of water and 30.7 parts by weight of 1-hexadecyl-3-vinyl-imidazol-1-ium bromide were mixed under nitrogen and heated to 60° C. 2.0 parts by weight of 2,2′-Azobis(2-methylpropionamidine)dihydrochloride (Wako V 50, Wako Chemicals GmbH, Germany) were mixed with 43.5 parts by weight of water and added within one hour at 60° C. During the same time 8.5 parts by weight of 2-isopropenyl-2-oxazoline were added. After addition of all components, the mixture was kept at 60° C. for 9 hours.

A milky white dispersion was obtained. 2-isopropenyl-2-oxazoline rest monomer content was <10 ppm.

### Example 3

### Preparation of Copolymer X3 (VP:iPDX 90:10)

271 parts by weight of water were heated under nitrogen to 60° C. 4.0 parts by weight of 2,2′-Azobis(2-methylpropionamidine)dihydrochloride (Wako V 50, Wako Chemicals GmbH, Germany) were mixed with 65 parts by weight of water and added within one hour at 60° C. During the same time 8.0 parts by weight of 2-isopropenyl-2-oxazoline and 72 parts by weight of vinyl pyrrolidone were added. After addition of all components, the mixture was kept at 60° C. for 9 hours. A slightly turbid solution was obtained. 2-isopropenyl-2-oxazoline rest monomer content was <20 ppm.

### Example 4

### Preparation of Copolymer X4 (VP:iPDX 80/20)

271 parts by weight of water were heated under nitrogen to 60° C. 4.0 parts by weight of 2,2′-Azobis(2-methylpropionamidine)dihydrochloride (Wako V 50, Wako Chemicals GmbH, Germany) were mixed with 65 parts by weight of water and added within one hour at 60° C. During the same time 16.0 parts by weight of 2-isopropenyl-2-oxazoline and 64 parts by weight of vinyl pyrrolidone were added. After addition of all components, the mixture was kept at 60° C. for 9 hours. A slightly turbid solution was obtained. 2-isopropenyl-2-oxazoline rest monomer content was <20 ppm.

### Example 5

### Preparation of Copolymer X5 (QVI-C12:iPDX 1:4)

319 parts by weight of water and 17.2 parts by weight of 1-dodecyl-3-vinyl-imidazol-1-ium bromide were mixed under nitrogen and heated to 60° C. 2.0 parts by weight of 2,2′-Azobis(2-methylpropionamidine)dihydrochloride (Wako V 50, Wako Chemicals GmbH, Germany) were mixed with 50.0 parts by weight of water and added within one hour at 60° C. During the same time 22.2 parts by weight of 2-isopropenyl-2-oxazoline were added. After addition of all components, the mixture was kept at 60° C. for 9 hours. A milky white dispersion was obtained. 2-isopropenyl-2-oxazoline rest monomer content was 30 ppm.

### Example 6

### Preparation of Copolymer X6 (QVI-C12:iPDX 1:2)

319 parts by weight of water and 17.2 parts by weight of 1-dodecyl-3-vinyl-imidazol-1-ium bromide were mixed under nitrogen and heated to 60° C. 2.0 parts by weight of 2,2′-Azobis(2-methylpropionamidine)dihydrochloride (Wako V 50, Wako Chemicals GmbH, Germany) were mixed with 50.0 parts by weight of water and added within one hour at 60° C. During the same time 11.1 parts by weight of 2-isopropenyl-2-oxazoline were added. After addition of all components, the mixture was kept at 60° C. for 9 hours. A milky white dispersion was obtained. 2-isopropenyl-2-oxazoline rest monomer content was 25 ppm.

### Example 7

### Preparation of Copolymer X7 (QVI-C16:iPDX 1:3)

319 parts by weight of water and 13.3 parts by weight of 1-hexadecyl-3-vinyl-imidazol-1-ium bromide were mixed under nitrogen and heated to 75° C. 1.2 parts by weight of 2,2′-Azobis(2-methylpropionamidine)dihydrochloride (Wako V 50, Wako Chemicals GmbH, Germany) were mixed with 63.0 parts by weight of water and added within one hour at 75° C. During the same time 11.1 parts by weight of 2-isopropenyl-2-oxazoline were added. After addition of all components, the mixture was kept at 75° C. for 9 hours.

A milky white dispersion was obtained. 2-isopropenyl-2-oxazoline rest monomer content was 5 ppm.

### Example 8

### Preparation of Copolymer X8 (Ralu®mer SPE:iPDX 90:10)

200 parts by weight of water were heated under nitrogen to 60° C. 4.0 parts by weight of 2,2′-Azobis(2-methylpropionamidine)dihydrochloride (Wako V 50, Wako Chemicals GmbH, Germany) were mixed with 37 parts by weight of water and added within one hour at 60° C. During the same time 8.0 parts by weight of 2-isopropenyl-2-oxazoline and a mixture consisting of 72 parts by weight of N-(2-Methacryloyloxyethyl)-N,N-dimethyl-N-(3-sulfopropyl)ammoniumbetaine (Ralu®mer SPE; Raschig GmbH, Germany) and 100 parts by weight of water were added. After addition of all components, the mixture was kept at 60° C. for 9 hours.

A slightly opaque yellow solution was obtained.

### Example 9

### Preparation of Copolymer X9 (QVI-C12:VP:iPDX 30:60:10)

400 parts by weight of water and 15 parts by weight of 1-dodecyl-3-vinyl-imidazol-1-ium bromide were mixed under nitrogen and heated to 60° C. 0.5 parts by weight of 2,2′-Azobis(2-methylpropionamidine)dihydrochloride (Wako V 50, Wako Chemicals GmbH, Germany) were mixed with 50.0 parts by weight of water and added within two hours at 60° C. Starting at the same time a mixture of 5.0 parts by weight of 2-isopropenyl-2-oxazoline and 30.0 parts by weight of N-vinyl pyrrolidone were added within one hour. After addition of all components, the mixture was kept at 60° C. for 6 hours. A clear yellow solution with some flakes was obtained.

### Example 10

### Preparation of Copolymer X10 (QVI-C12:VP:iPDX 20:70:10)

400 parts by weight of water and 10 parts by weight of 1-dodecyl-3-vinyl-imidazol-1-ium bromide were mixed under nitrogen and heated to 60° C. 0.5 parts by weight of 2,2′-Azobis(2-methylpropionamidine)dihydrochloride (Wako V 50, Wako Chemicals GmbH, Germany) were mixed with 50.0 parts by weight of water and added within two hours at 60° C. Starting at the same time a mixture of 5.0 parts by weight of 2-isopropenyl-2-oxazoline and 35.0 parts by weight of N-vinyl pyrrolidone were added within one hour. After addition of all components, the mixture was kept at 60° C. for 6 hours. An opaque viscous solution was obtained.

### Example 11

### Preparation of Copolymer X11 (BAEMA:VP:iPDX 30:60:10)

240 parts by weight of water were heated to 60° C. under nitrogen. 0.5 parts by weight of 2,2′-Azobis(2-methylpropionamidine)dihydrochloride (Wako V 50, Wako Chemicals GmbH, Germany) were mixed with 45.0 parts by weight of water and added within two hours at 60° C. Starting at the same time a mixture of 5.0 parts by weight of 2-isopropenyl-2-oxazoline and 30.0 parts by weight of N-vinyl pyrrolidone were added within one hour. Also starting at the same time 15.0 parts by weight of 2-tert-butylaminoethyl methacrylate were added within one hour. After addition of all components, the mixture was kept at 60° C. for 6 hours. A clear solution was obtained.

### Example 12

### Preparation of Copolymer X12 (TBAEMA:VP:iPDX 60:30:10)

340 parts by weight of water were heated to 60° C. under nitrogen. 0.5 parts by weight of 2,2′-Azobis(2-methylpropionamidine)dihydrochloride (Wako V 50, Wako Chemicals GmbH, Germany) were mixed with 45.0 parts by weight of water and added within two hours at 60° C. Starting at the same time a mixture of 5.0 parts by weight of 2-isopropenyl-2-oxazoline and 15.0 parts by weight of N-vinyl pyrrolidone were added within one hour. Also starting at the same time 30.0 parts by weight of 2-tert-butylaminoethyl methacrylate were added within one hour. After addition of all components, the mixture was kept at 60° C. for 6 hours. A clear solution was obtained.

### Example 14

### Preparation of reverse osmosis membranes

Support ultrafiltration membranes were first stored overnight (>12 h) in deionized water. Afterwards the membrane surface was treated with a rubber roller to remove water droplets and the membrane was fixed in a frame structure (PMMA plate and a silicone and PMMA frame). An aqueous 1.5-2% (w/v) m-Phenylenediamine solution (deionized water) and 0.025 to 1.3% (w/v) Trimesoyl chloride solution in dry dodecane were prepared. 50 ml of the m-Phenylenediamine solution was poured into the frame construction onto the membrane surface. The exposure time was 10 min. After pouring off the m-Phenylenediamine solution and disassembling the frame construction, the wetted membrane was placed on a PMMA plate covered with a paper towel. With a rubber roller solution droplets were gently removed from the membrane surface. The tissues were removed and the membrane was clamped in the frame construction again. Now the one-minute polycondensation reaction was initiated by adding 50 ml of 0.025 to 1.3% (w/v) Trimesoyl chloride solution. The Trimesoyl chloride solution was poured out of the frame construction and the frames were disassembled. In order to remove residual monomer solution from the membrane surface, the membrane was rinsed with 75 ml of n-hexane on the PMMA plate in a tilted position. The membrane was placed down to evaporate hexane for one minute. The thin film composite membrane with the gleaming polyamide layer was finally stored in deionized water for 24 h.

### Example 15

### Coating of a RO membrane with oxazoline-containing copolymers

On a RO membrane comprising polyamide as the main component in the separation layer a thin layer of one of the aqueous copolymer solutions X1-X12 described in examples 1 to 12 is applied by use of a draw-down bar of 15 μm, 100 μm or 200 μm slit width at a speed of 25 mm/s. The copolymer solutions are either undiluted or diluted to a solid content of 1% or 0.1% w/w. The membrane is heated to 80° C. for drying the film.

### Example 16

### Coating of a RO Membrane with Oxazoline-Containing Copolymers and Ammonium Polyacrylate

One of the aqueous copolymer solutions X1-X12 described in examples 1 to 12 is mixed with polyacrylic acid ammonium salt aqueous solution (Dispex® AA 4040, BASF SE) such that equimolar amounts of carboxylic acid and 2-isopropenyl-2-oxazoline groups are present. A thin layer of this mixture is applied by use of a draw-down bar of 15 μm, 100 μm or 200 μm slit width at a speed of 25 mm/s to an RO membrane comprising polyamide as the main component in the separation layer. The copolymer solutions are either undiluted or diluted to a solid content of 1% or 0.1% w/w. The membrane is heated to 80° C. for drying the film.

### Example 17

### Protein Adhesion on a RO Membrane Coated with Copolymer X2

A RO membrane is coated with Copolymer X2 and tested for protein adhesion as described above. Protein adhesion to the coated membrane is reduced by approximately 60% as compared to an uncoated membrane.

### Example 18

### Protein Adhesion on a RO Membrane Coated with Copolymer X3

A RO membrane is coated with Copolymer X3 and tested for protein adhesion as described above. Protein adhesion to the coated membrane is reduced by approximately 85% as compared to an uncoated membrane.

### Example 19

### Bacterial Adhesion on a RO Membrane Coated with Copolymer X2

A RO membrane is coated with Copolymer X2 and tested for bacterial adhesion as described above. Bacteria adhesion to the coated membrane is reduced by approximately 90% as compared to an uncoated membrane.

### Example 20

### Bacterial Adhesion on a RO Membrane Coated with Copolymer X3

A RO membrane is coated with Copolymer X3 and tested for bacterial adhesion as described above. Bacteria adhesion to the coated membrane is reduced by approximately 98% as compared to an uncoated membrane.

### Example 21

### Antimicrobial Activity of a RO Membrane Coated with Copolymer X2

A RO membrane is coated with Copolymer X2 and tested for antimicrobial activity using fluorescence microscopy as described above. Approximately 95% of adherent bacteria are inactivated.

### Example 22

### Antimicrobial Activity of a RO Membrane Coated with Copolymer X2 According to ISO 22196 (JIS Z2801)

RO membranes are coated with Copolymer X2 and tested for antimicrobial activity according to ISO 22196 (JIS Z2801). Average reductions of colony forming units as compared to blank membrane controls of >3 log units for S. aureus DSM 346 and of >5 log units for E. coli DSM 1576 are found.

### Example 23

### Antimicrobial Activity of a RO Membrane Coated with Copolymer X5 According to ISO 22196 (JIS Z2801)

RO membranes are coated with Copolymer X5 and tested for antimicrobial activity according to ISO 22196 (JIS Z2801). Average reductions of colony forming units as compared to blank membrane controls of >5 log units for S. aureus DSM 346 and of >5 log units for E. coli DSM 1576 are found.

### Example 24

### Antimicrobial Activity of a RO Membrane Coated with Copolymer X6 According to ISO 22196 (JIS Z2801)

RO membranes are coated with Copolymer X9 and tested for antimicrobial activity according to ISO 22196 (JIS Z2801). Average reductions of colony forming units as compared to blank membrane controls of >5 log units for S. aureus DSM 346 and of >5 log units for E. coli DSM 1576 are found.

### Example 25

### Antimicrobial Activity of a RO Membrane Coated with Copolymer X10 According to ISO 22196 (JIS Z2801)

RO membranes are coated with Copolymer X7 and tested for antimicrobial activity according to ISO 22196 (JIS Z2801). Average reductions of colony forming units as compared to blank membrane controls of >5 log units for S. aureus DSM 346 and of >5 log units for E. coli DSM 1576 are found.

1: A polymer, comprising
i) an oxazoline of formula 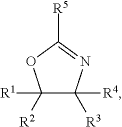
wherein R¹, R², R³ and R⁴ each independently denote a hydrogen atom, a halogen atom, an alkyl group, an aralkyl group, a phenyl group, or a substituted phenyl group, and R⁵ denotes a noncyclic organic group having an unsaturated bond reactive in radical polymerization,
ii) optionally an antiadhesive monomer,
iii) optionally a biocidal monomer, and
iv) optionally a further monomer,
wherein the polymer comprises at least one monomer selected from monomers ii) and iii),
wherein monomers ii) are selected from
a) esters of (meth)acrylic acid polyols, wherein (meth)acrylic esters with polyalkyleneoxides are excluded;
b) vinyl ethers of polyols;
c) hydrophilic macromonomers different from monomers a) and b);
d) vinyl compounds;
e) low molecular weight hydrophilic (meth)acrylamides;
f) (meth)acrylates or (meth)acrylamides bearing epoxy groups;
g) monomers having a betain structure;
h) hydrophilic monomers selected from hydroxyethyl-(meth)acrylate, vinyl alcohol, (Meth)acryloyl and (meth)acrylamide-modified mono- and oligosaccharides, 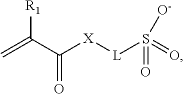
wherein R1=H or Methyl and X═O or NH; and
i) ion pair comonomers;
and wherein monomers iii) are selected from
j) vinyl-imidazolium compounds;
k) ethylenically unsaturated monomers bearing quarternary ammonium or phosphonium groups;
l) diallyldialkylammoniumchlorides;
m) alkylaminoalkyl(meth)acrylates and alkylaminoalkyl(meth)acrylamides;
n) polylysine(meth)acrylamides or (meth)acrylates;
o) alkyl-4-vinylpridinium and alkyl-2-vinyl-pyridinium salts;
p) ethylenically unsaturated monomers bearing guanide and biguanide groups; and
q) halamines.

2: The polymer according to claim 1, wherein the oxazoline is at least one selected from the group consisting of 2-Isopropenyl-2-oxazolin, 2-vinyl-2-oxazoline, 2-vinyl-4-methyl-2-oxazoline, 2-vinyl-5-methyl-2-oxazoline, 2-isopropenyl-4-methyl-2-oxazoline and 2-isopropenyl-5-ethyl-2-oxazoline.

3: The polymer according to claim 1, wherein the oxazoline is 2-Isopropenyl-2-oxazoline.

4: The polymer according to claim 1, wherein the polymer comprises monomers ii) and iii) in an amount of from 5 to 95% by weight relative to the polymer.

5: The polymer according to claim 1, wherein the polymer comprises a monomer of formula (I) 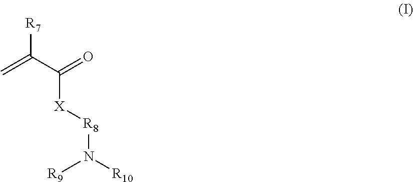
wherein
R₇ is H or CH₃,
R₈ is C₁-C₅ alkyl bi-radical,
R₉ and R₁₀ are each independently H or C₁-C₅ alkyl radical which can be linear or branched,
and X is a divalent radical of —O—, —NH— or —NR₁₁, wherein R₁₁ is C₁-C₆ alkyl.

6: Polymer according to claim 1, wherein said polymer comprises 2-tert-butylaminoethyl(meth)acrylate or a combination of 2-Hydroxyethyl methacrylate and [2-(methacryloyloxy)ethyl]trimethylammonium chloride.

7: A method for imparting biocidal and/or antiadhesive properties to a surface, comprising coating the surface with the polymer according to claim 1.

8: A membrane, comprising a polymer, which comprises
i) an oxazoline of formula 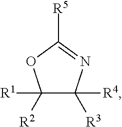
wherein R¹, R², R³ and R⁴ each independently denote a hydrogen atom, a halogen atom, an alkyl group, an aralkyl group, a phenyl group, or a substituted phenyl group, and R⁵ denotes a noncyclic organic group having an unsaturated bond reactive in radical polymerization,
ii) optionally an antiadhesive monomer,
iii) optionally a biocidal monomer, and
iv) optionally a further monomer,
wherein the polymer comprises at least one monomer selected from monomers ii) and iii),
wherein monomers ii) are selected from
a) esters of (meth)acrylic acid polyols, wherein (meth)acrylic esters with polyalkyleneoxides are excluded;
b) vinyl ethers of polyols;
c) hydrophilic macromonomers different from monomers a) and b);
d) N-vinyl compounds;
e) low molecular weight hydrophilic (meth)acrylamides;
f) (meth)acrylates or (meth)acrylamides bearing epoxy groups;
g) monomers having a betain structure;
h) hydrophilic monomers selected from hydroxyethyl-(meth)acrylate, vinyl alcohol, (Meth)acryloyl and (meth)acrylamide-modified mono- and oligosaccharides, 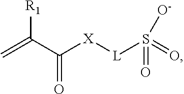
wherein R1=H or Methyl and X═O or NH; and
i) ion pair comonomers;
and wherein monomers iii) are selected from
j) vinyl-imidazolium compounds;
k) ethylenically unsaturated monomers bearing quarternary ammonium or phosphonium groups;
l) diallyldialkylammoniumchlorides;
m) alkylaminoalkyl(meth)acrylates and alkylaminoalkyl(meth)acrylamides;
n) polylysine(meth)acrylamides or (meth)acrylates;
o) alkyl-4-vinylpridinium and alkyl-2-vinyl-pyridinium salts;
p) ethylenically unsaturated monomers bearing guanide and biguanide groups; and
q) halamines.

9: The membrane according to claim 8, wherein the oxazoline is 2-Isopropenyl-2-oxazoline.

10: The membrane according to claim 8, wherein the polymer comprises monomers ii) and iii) in an amount of from 5 to 95% by weight relative to the polymer.

11: The membrane according to claim 8, wherein the polymer comprises a monomer of formula (I) 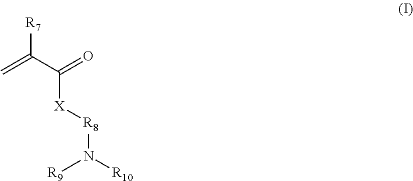
wherein
R₇ is H or CH₃,
R₈ is C₁-C₅ alkyl bi-radical,
R₉ and R₁₀ are each independently H or C₁-C₅ alkyl radical which can be linear or branched,
and X is a divalent radical of —O—, —NH— or —NR₁₁, wherein Rₗ 1 is C₁-C₆ alkyl.

12: The membrane according to claim 8, wherein the polymer comprises 2-tert-butylaminoethyl(meth)acrylate or a combination of 2-Hydroxyethyl methacrylate and [2-(methacryloyloxy)ethyl]trimethylammonium chloride.

13: The membrane according to claim 8, wherein the polymer has been coated or grafted on the outermost layer of the membrane facing the feed side of the membrane.

14: The membrane according to claim 8, wherein the membrane or the separating layer of the membrane comprises as the main component a polymer selected from the group consisting of polyarylene ether, polysulfone, polyethersulfones (PES), polyphenylenesulfone, polyamides (PA), polyvinylalcohol (PVA), cellulose acetate (CA), cellulose triacetate (CTA), CA-triacetate blend, cellulose ester, cellulose nitrate, regenerated cellulose, aromatic, aromatic/aliphatic or aliphatic polyamide, aromatic, aromatic/aliphatic or aliphatic polyimide, polybenzimidazole (PBI), polybenzimidazolone (PBIL), polyacrylonitrile (PAN), PAN-poly(vinyl chloride) copolymer (PAN-PVC), PAN-methallyl sulfonate copolymer, polysulfone, poly(dimethylphenylene oxide) (PPO), polycarbonate, polyester, polytetrafluroethylene PTFE, poly(vinylidene fluoride) (PVDF), polypropylene (PP), polyelectrolyte complexes, poly(methyl methacrylate) PMMA, polydimethylsiloxane (PDMS), aromatic, aromatic/aliphatic or aliphatic polyimide urethanes, aromatic, aromatic/aliphatic or aliphatic polyamidimides, crosslinked polyimides and mixtures thereof.

15: The membrane according to claim 8, in which the membrane is suitable as a reverse osmosis membrane, forward osmosis membrane, nanofiltration membrane, ultrafiltration membrane and/or microfiltration membrane.

16: A process for making a membrane according to claim 8, comprising coating the surface of a base membrane with a formulation comprising:
A) coating the surface of a base membrane with a formulation comprising
I) the polymer,
II) optionally at least one di- or polycarboxylic acid, di- or polysulfonic acid, di- or polyphosphonic acid, di- or poly phosphoric acid or components comprising two or more of these acid groups and/or thiol groups or latent acids, di- or polyacids that form the acid during the coating process;
B) optionally annealing of the coating; and
C) optionally extracting nonreacted components from I) and/or II).

17: The process according to claim 16, further comprising:
D) surface of modifying the base membrane with at least one oxidative process to obtain anchor groups; and/or
E) surface of modifying the base membrane with a nonoxidative process to obtain anchor groups.

18: The process according to claim 16, further comprising treating a base membrane with a formulation comprising components I, wherein the formulation does not comprise a component II.

19: The membrane according to claim 8, wherein the membrane is suitable for the treatment of water, for the desalination of sea water or brackish water, for the treatment of industrial or municipal wastewater in food processing, or medical applications like dialysis.
